(19) United States
(12) Patent Application Publication
(10) Pub. No.: US 2023/0029751 A1
(43) Pub. Date: Feb. 2, 2023

(54) CLASSIFYING TUMORS USING BOTH GENETICS AND COLLAGEN EXPRESSION TO IMPROVE DRUG TARGETING

(71) Applicant: Rhode Island Hospital, Providence, RI (US)

(72) Inventors: Alexander S. BRODSKY, Newton, MA (US); Kevin S. GUO, Staten Island, NY (US)

(21) Appl. No.: 17/814,400

(22) Filed: Jul. 22, 2022

Related U.S. Application Data
(60) Provisional application No. 63/224,530, filed on Jul. 22, 2021.

(51) Int. Cl. G16B 25/10; G01N 33/574; C12Q 1/6886

(52) U.S. Cl. CPC G16B 25/10; G01N 33/574; C12Q 1/6886; C12Q 2600/112; G01N 2800/60; C12Q 2600/158; C12Q 2600/156; C12Q 2600/106

(57) ABSTRACT

The invention provides a method for classifying tumors by their collagen expression patterns into groups associated with high and low overall survival.

### REFERENCE TO RELATED APPLICATIONS

This document claims the benefit of priority to patent application U.S. Ser. No. 63/224,530, filed Jul. 22, 2021, the entire contents of which are incorporated by reference herein.

### TECHNICAL FIELD OF THE INVENTION

This invention generally relates to classifying tumors using both genetics and collagen expression to improve drug targeting.

### BACKGROUND OF THE INVENTION

Matching therapies to specifics of a patient's tumor make-up remains one of the great outstanding challenges in oncology. Many targeted treatments have low or modest objective responses such that many patients do not benefit from the treatment based on current biomarkers and genetic analysis.

Although the relationship between genotype and the tumor environment remains unclear, tumors evolve in specific contexts such that the genotypes and tumor environment shape each other to create an ecosystem.

Successful personalized medicine requires tumor classification that predicts patient responses with high accuracy. Letai, Bhola, & Welm, Cancer Cell (2021). The medical hope of targeting the same pathway has been challenged by the poor predictability of many treatments across anatomic classifications. See Letai, Bhola, & Welm, Cancer Cell (2021).

There is a need in the biomedical art to improve tumor classification by considering the whole tumor ecosystem.

### SUMMARY OF THE INVENTION

The invention improves tumor classification by considering the whole tumor ecosystem. The invention combines genotypes with components of the extracellular matrix (ECM) to develop new prognostic markers and improve targeting treatments.

In a first embodiment, the invention provides a method for classifying tumors by their collagen expression patterns into groups associated with high and low overall survival. These groups show strong biases to the tumor genetics, including somatic mutations, ploidy, and aneuploidy status. This approach classifies tumors into groups with specific genetic signatures with long and short survival that address how patients with tumors with similar genetic profiles show a wide range of responses and overall survival. The tumor extracellular matrix dictates how tumors respond to treatment.

This invention uses collagens to classify tumors to improve prognosis and diagnosis. Classification by collagens incorporates aspects of the tumor context to better classify tumors with their genetics.

When each of the solid tumor cancer types in The Cancer Genome Atlas (TCGA) was clustered by the RNA expression of the forty-three collagen genes, the inventors made strong associations with overall survival, specific immunoenvironments, somatic gene mutations, copy number variations, and aneuploidy.

Matrisome and collagen RNA expression based clustering PanCancer grouped tumors by their tissue of origin.

In a second embodiment, the invention provides a method for treating cancer in a subject. The first step of the method is selecting a tumor classification associated with high and low overall survival for a tumor by its collagen expression patterns into groups. Predicting patient response to treatment should consider both tumor collagen composition and genetics.

The second step of the method is treating the subject with a cancer treatment specific for the tumor classification associated with high and low overall survival. The invention provides therapies including immunotherapy, targeted therapy, and chemotherapy better tailored to individual subjects by considering the collagen and matrisome milieu.

This method is useful for several cancer types, including bladder urothelial carcinoma (BLAC); breast invasive carcinoma (BRAC); endocervical adenocarcinoma (CESC); colon adenocarcinoma (COAD); colorectal carcinoma (COADREAD); esophageal carcinoma (ESCA); glioblastoma multiforme (GBM); head and neck squamous cell carcinoma (HNSC); kidney renal clear cell carcinoma (KIRC); kidney renal papillary cell carcinoma (KIRP); brain lower grade glioma (LGG); liver hepatocellular carcinoma (LIHC); lung adenocarcinoma (LUAD); lung squamous cell carcinoma (LUSC); ovarian serous cystadenocarcinoma (OV); pancreatic adenocarcinoma (PAAD); pheochromocytoma and paraganglioma (PCPG); prostate adenocarcinoma (PRAD); rectal adenocarcinomas (READ); sarcoma (SARC); skin cutaneous melanoma (SKCM); stomach adenocarcinoma (STAD); testicular germ cell tumors (TGCT); thyroid carcinoma (THCA); thyoma (THYM); and uterine corpus endometrial carcinoma (UCEC).

In a third embodiment, the invention provides a machine learning classifier that predicted a tumor's aneuploidy, KRAS mutation, Myc amplification or chromosome arm copy number alteration (CNA) status based on only collagen RNA expression with high accuracy in many cancer types, showing a strong relationship between the extracellular matrix context and specific molecular alterations.

The Support Vector Machine (SVM) models predicted specific molecular alterations based only on collagen expression. The findings provided by the machine learning classifier have broad implications in defining the relationship between molecular alterations and the tumor microenvironment to improve prognosis and therapeutic targeting for patient care, opening new avenues of investigation to define tumor ecosystems.

The approach is to analyze The Cancer Genome Atlas dataset both PanCancer and within each cancer type individually.

In one aspect, the invention provides an analysis of collagen composition in individual cancer types and PanCancer to improve tumor classification and gain new insights into the relationship between the tumor extracellular matrix and cancer genome.

Classifying with just collagens is similar to classifying with the full matrisome. We demonstrated that collagen composition is distinct in most cancer types. This highlights how the extracellular matrix is linked to the lineage and tissue of origin. Defining tissue by the extracellular matrix composition stresses how tissue is defined by the milieu holding cells together, reflecting the complex interplay of myriad cells in each tissue. The Support Vector Machine models predicting molecular alterations from collagen RNA expression provide further evidence of specific relationships between the extracellular matrix and the cancer genome. The strongest links across multiple cancer types were between collagen expression and global features such as aneuploidy. All together, these findings indicate that cancer cell state is associated with specific collagen defined extracellular matrices implying that the extracellular matrix state is critical factor to properly target tumors.

The invention takes advantage of the context specific collagen expression leading to the identification of the context specific, clinically actionable enrichment of drivers and established biomarkers including copy number alterations in ColClusters.

The tumor extracellular matrix and collagen composition reflect the contributions of fibroblasts, macrophages and other cells that all secrete collagens to create the complex tumor tissue structure. Because the extracellular matrix and collagen composition results from a complicated mixture of cells both secreting and remodeling, an extracellular matrix-collagen based classifier may gain its power because it is the sum of the output of the ecosystem, reflecting both cell composition and cell states. These observations show that classifying tumors by extracellular matrix composition is likely beneficial to capture the past origins and future fate of disease progression. Classifying and targeting aneuploid tumors remains a major challenge. Collagen clustering through both enrichment and machine learning prediction approaches show a connection between the genome architecture and the surrounding milieu. In many cancer types, aneuploidy combined with collagen composition identify tumor classes associated with overall survival not uncovered when considering aneuploidy tumors by themselves. Some groups, such as in lung squamous cell carcinoma (LUSC), identify tumor groups where aneuploid tumors had relatively higher or lower overall survival depending on their collagen composition.

These correlative classifications are not meant to definitive exclusive relationships, which definitiveness would not be accepted by persons having ordinary skill in the biomedical art. These classifications may instead be understood by persons having ordinary skill in the biomedical art to encompass the actively changing biologically transcriptional states captured by classification approaches.

In one aspect, the invention directly considers the microenvironment classifies tumors and reveals putative relationships between molecular alterations, transcriptional states and the extracellular matrix. The association data in this study cannot discriminate between the possible mechanisms behind the observations. There are two likely scenarios: the collagen environment may select for specific cancer genomes or specific cancer genomes may remodel the collagen environment to fit its needs over other clones. Further study could test these hypotheses to untangle the relationship in other patient cohorts and in pre-clinical models. Although PanCancer studies can be informative to identify general principles of tumors, they suffer from the averaging of many of the tissue specific features likely critical for targeting tumors.

By organizing tumors by their tissue of origin, the inventors identified specific features of the extracellular matrix associated with genotypes and phenotypes useful for personalizing targeting.

Cancer cells are selected for specific properties and genomes in different collagen defined tumor extracellular matrices and that the whole panoply of collagens contribute to the extracellular matrix and tumor evolution.

Collagens are useful biomarkers of the tumor ecosystem and disease progression.

### BRIEF DESCRIPTION OF THE DRAWINGS

For illustration, some embodiments of the invention are shown in the drawings described below. Like numerals in the drawings indicate like elements throughout. The invention is not limited to the precise arrangements, dimensions, and instruments shown.

FIG. 1 is a graph showing collagen clusters associated with overall survival. Example: stomach adenocarcinoma.

FIG. 2 is a bubble plot showing aneuploid genomes associated with specific collagen environments. FIG. 2 is a bubble plot of aneuploidy scores in each collagen cluster normalized relative to ColCluster-1 for each cancer type.

FIG. 3 is a schema showing aneuploid genomes associated with specific collagen environments. Collagens can classify tumors by tissue type. Many collagens are specific in tissue. Dysregulation of collagens further defines the lineage and tumor groups. The overlap with reported PanCancer tissue typing and histology was good.

FIG. 4 is a set (FIG. 4A-FIG. 4Z) of diagrams showing decision trees based upon the results of this specification.

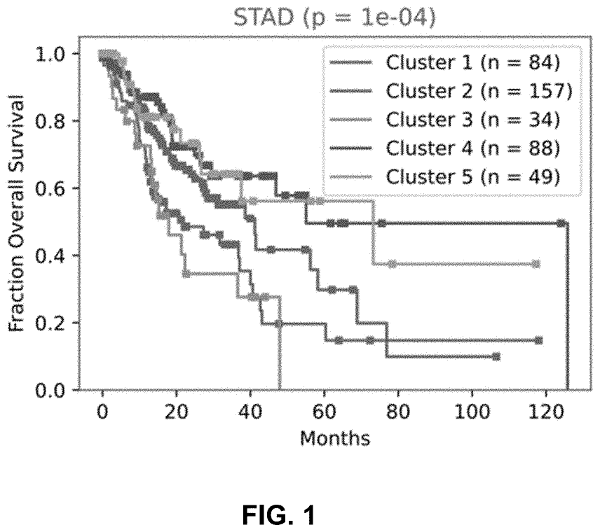

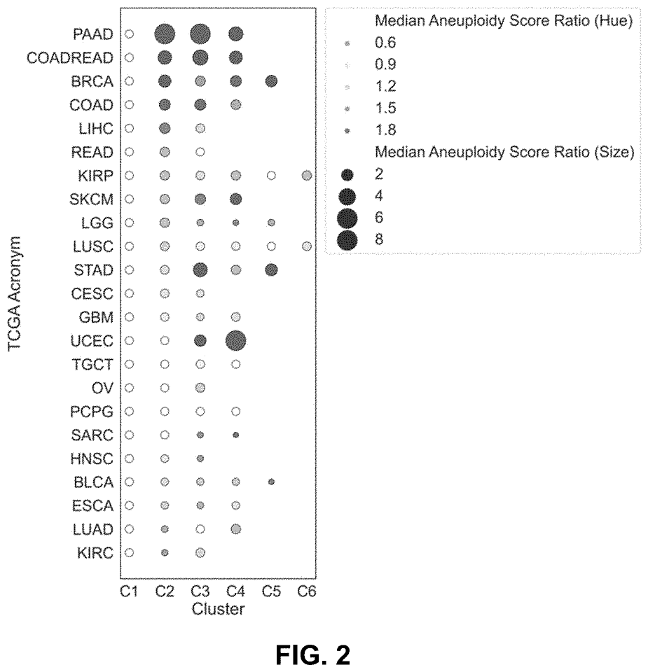

### DETAILED DESCRIPTION OF THE INVENTION

### Industrial Applicability

The findings in this specification can be developed into a clinical test measuring collagen RNA and protein expression. The approach can be extended to include the entire matrisome to refine further and increase the robustness of the classifier. The inventors tested the classifier across multiple cancer types using publicly available data.

Diagnostic test companies can develop a diagnostic test based on our findings.

Pathologists and oncologists can use such a test to improve drug choices for cancer patients. Biotech and pharma companies could use this approach to help drug development and tailor therapies to specific tumor classes defined by collagens.

### Introduction

Molecular targeting has not typically considered the tumor extracellular matrix (ECM) when considering therapy options. The extracellular matrix is a collection of structural proteins and enzymes that holds the cells together. The tumor extracellular matrix influences tumor growth, metastasis, and patient outcomes, in part through regulation of the cancer hallmarks. Pickup et al., (2014). The tumor microenvironment is increasingly being demonstrated to impact cell states, therapy responses, and patient outcomes.

High expression of collagens in tumors has long been associated with poor outcomes as part of stromal expression signatures in many, but not all, cancer types Farmer et al., (2009); Brodsky et al., (2014). These stroma, or mesenchymal, groups are enriched for collagens, but the expression of collagens in tumors has not been systematically evaluated.

Previous studies have evaluated aspects of the matrisome in The Cancer Genome Atlas showing that an organized transcription factor network specifies the extracellular matrix. Izzi et al., Matrix Biology Plus (2019). Proteomics is revealing the complexity of the matrisome originating from multiple cell types (Tian et al., (2020, 2021)). Individual collagens such as collagen types IV, (Lindgren et al., (2021)), collagen type X, and XI (Nallanthighal et al., (2021)) have been proposed as biomarkers. These findings emphasize the importance of the matrisome and collagens in forming the tumor ecosystem. Because collagens and the matrisome proteins are secreted from multiple cell types, the extracellular matrix composition reflects the output of myriad cell types and pathways summing to influence disease progression.

Many pathways and molecular alterations have context dependent impacts complicating therapeutic decision-making. the inventors hypothesized that tumors can be classified by their extracellular matrix composition revealing connections among pathways, molecular alterations and the microenvironment. Collagens constitute up to 30% of the total protein in the body and are the major components of the extracellular matrix. The inventors found that classifying tumors by just the expression of the forty-three collagen genes captures the seminal features compared to classifying a large set of hundreds of genes representing the matrisome and simplifies analysis to demonstrate specificity. Collagen defined classification in multiple cancer types identified strong associations with overall survival, pathways, molecular alterations, histology, and the tissue of origin. Collagen clustering classified tumors with aneuploidy into distinct groups associated with overall survival in multiple cancer types and machine learning predicted aneuploidy, copy number alterations and other molecular alterations from just collagen expression. Similarly, enrichment of specific somatic mutations by collagen classification implies that the combination of the genetics and collagen tumor environment may improve therapeutic targeting. These observations highlight the importance of the composition of the tumor extracellular matrix in mediating the impact of molecular alterations and the immunoenvironment to guide therapy.

### Anticancer Therapies

Many therapeutically useful anticancer therapies are known in the biomedical art. Many therapeutically useful anticancer therapies are known for specific kinds of cancers.

Chemotherapy means the administration of any chemical agent with therapeutic usefulness in the treatment of diseases characterized by abnormal cell growth. Such diseases include tumors, neoplasms, and cancer and diseases characterized by hyperplastic growth. Chemotherapeutic agents encompass both chemical and biological agents. These agents function to inhibit a cellular activity upon which the cancer cell depends for continued survival. Categories of chemotherapeutic agents include alkylating/alkaloid agents, antimetabolites, hormones or hormone analogs, and other antineoplastic drugs. Most of these agents are directly toxic to cancer cells and do not require immune stimulation. In one embodiment, a chemotherapeutic agent is an agent of use in treating neoplasms such as solid tumors. In one embodiment, a chemotherapeutic agent is a radioactive molecule. One of skill in the art can readily identify a chemotherapeutic agent of use (e.g., see Slapak & Kufe, Principles of Cancer Therapy, Chapter 86 in Harrison's Principles of Internal Medicine, 14th edition; Perry et al., Chemotherapy, Ch. 17 in Abeloff, Clinical Oncology 2nd Edition (Churchill Livingstone, Inc 2000); The bispecific and multispecific polypeptide agents can be used with additional chemotherapeutic agents.

The actual dosage levels of the T-cell, drug, and vaccine therapeutics in the pharmaceutical compositions of the invention may be varied to obtain an amount of the T-cell, drug and vaccine therapeutics which are effective to achieve the desired therapeutic response for a particular patient, composition, and mode of administration, without being toxic to the patient. The selected dosage level will depend upon a variety of pharmacokinetic factors including the activity of the particular compositions of the invention used, the route of administration, the time of administration, the rate of excretion of the particular compound being used, the duration of the treatment, other drugs, compounds and/or materials used combined with the particular compositions used, the age, sex, weight, condition, general health and prior medical history of the patient being treated, and like factors well known in the medical arts.

The pharmaceutical composition may be administered by any suitable route and mode.

CAR-T cell and related therapies relate to adoptive cell transfer of immune cells (e.g., T cells) expressing a CAR that binds specifically to a targeted cell type (e.g., cancer cells) to treat a subject. The cells administered as part of the therapy can be autologous to the subject. The cells administered as part of the therapy are not autologous to the subject. The cells are engineered or genetically modified to express the CAR. Further discussion of CAR-T therapies can be found, e.g., in Maus et al., Blood 123, 2624-35 (2014); Reardon et al., Neuro-Oncology, 16, 1441-1458 (2014); Hoyos et al., Haematologica 2012 97, 1622; Byrd et al., J Clin Oncol 2014 32, 3039-47; Maher et al., Cancer Res 2009 69, 4559-4562; and Tamada et al., Clin Cancer Res 2012 18, 6436-6445.

### Definitions

For convenience, the meaning of some terms and phrases used in the specification, examples, and appended claims, are listed below. Unless stated otherwise or implicit from context, these terms and phrases shall have the meanings below. These definitions aid in describing particular embodiments but are not intended to limit the claimed invention. Unless otherwise defined, all technical and scientific terms have the same meaning as commonly understood by a person having ordinary skill in the art to which this invention belongs. A term's meaning provided in this specification shall prevail if any apparent discrepancy arises between the meaning of a definition provided in this specification and the term's use in the biomedical art.

About has the plain meaning of approximately. The term about encompasses the measurement errors inherently associated with the relevant testing. When used with percentages, about means±1%. About or approximately when referring to a value or parameter means to be within a range of normal tolerance in the art, e.g., within two standard deviations of the mean. A description referring to about X includes description of X.

Activated CD8 T cells has the biomedical art-recognized meaning.

Activated Dendritic Cells (aDC) has the biomedical art-recognized meaning.

Adipogenesis has the biomedical art-recognized meaning of the formation of adipocytes (fat cells) from stem cells. Adipogenesis has two phases, determination and terminal differentiation.

Administering has the medical art-recognized meaning of placing a therapeutic composition of matter into or onto a subject's body by a method or route which results in at least partial delivery of the agent at a desired site. Administering can be by applying, ingesting, inhaling, or injecting a therapeutic composition of matter to or by a subject. The administration of the therapeutic composition of matter can be by any convenient manner

Argonaute RISC catalytic component 2 (AGO2) has the biomedical art-recognized meaning. The protein is required for RNA-mediated gene silencing (RNAi) by the RNA-induced silencing complex (RISC).

Allograft Rejection has the biomedical art-recognized meaning.

Aneuploidy has the biomedical art-recognized meaning of occurrence of one or more extra or missing chromosomes leading to an unbalanced chromosome complement, or any chromosome number that is not an exact multiple of the human haploid number (which is 23). See National Cancer Institute (NCI) Dictionary of Cancer Terms.

Angiogenesis has the biomedical art-recognized meaning of the development of new blood vessels.

Adenomatous polyposis coli (APC), also known as deleted in polyposis 2.5 (DP2.5) has the biomedical art-recognized meaning of a protein that in humans is encoded by the APC gene. The APC protein is a negative regulator that controls beta-catenin concentrations and interacts with E-cadherin, which are involved in cell adhesion. The APC gene encodes a multidomain protein that functions in tumor suppression by antagonizing the WNT signaling pathway.

Apical Surface has the biomedical art-recognized meaning. The apical surface of epithelial cells, which lines the lumen of sac- and tube-shaped organs and the inner surfaces of the body cavities, forms the interface between the extracellular milieu and underlying tissues.

AT-Rich Interaction Domain 1A (ARID1A) has the biomedical art-recognized meaning. ARID1A is a member of the SWI/SNF family, whose members have helicase and ATPase activities and are thought to regulate transcription of certain genes by altering the chromatin structure around those genes.

AUC means Area Under the Curve, a statistical measurement. Area under the curve is calculated by different methods known to persons having ordinary skill in the biomedical art.

Biomarker has the definition of biomarkers provided by the by the World Health Organization could be used: The International Programme on Chemical Safety, led by the World Health Organization (WHO) in coordination with the United Nations and the International Labor Organization, has defined a biomarker as ‘any substance, structure, or process that can be measured in the body or its products and influence or predict the incidence of outcome or disease.’ WHO International Programme on Chemical Safety Biomarkers in Risk Assessment: Validity and Validation (2001). See also Strimbu & Tavel, What are Biomarkers? Curr. Opin. HIV AIDS, 5(6): 463-466 (November 2011).

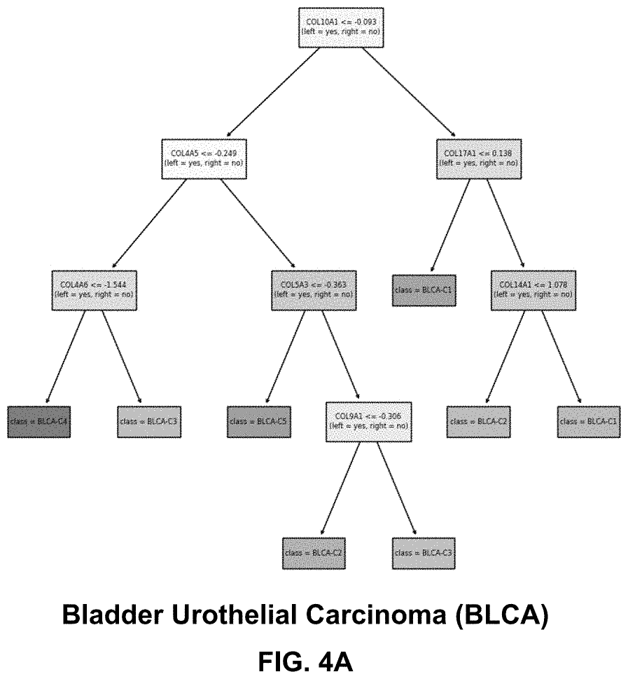

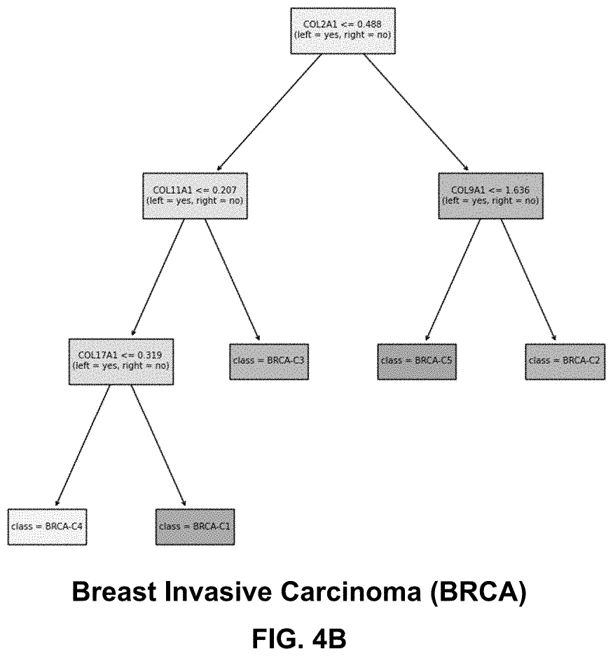

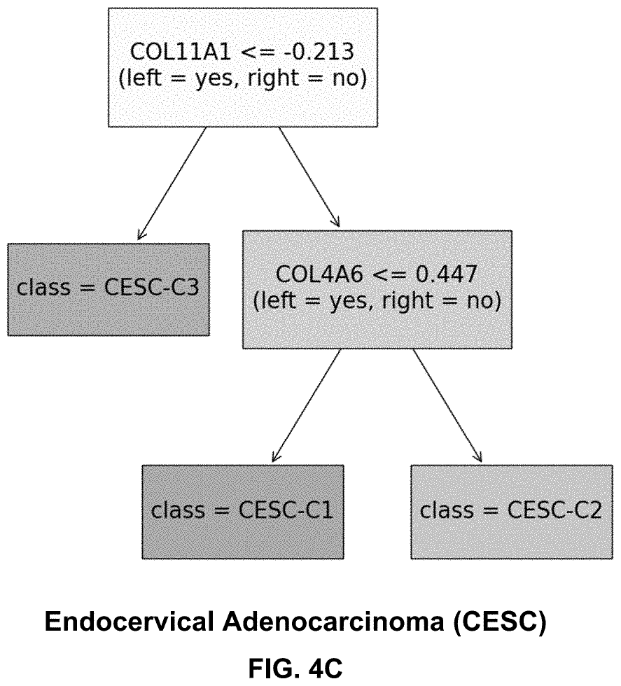

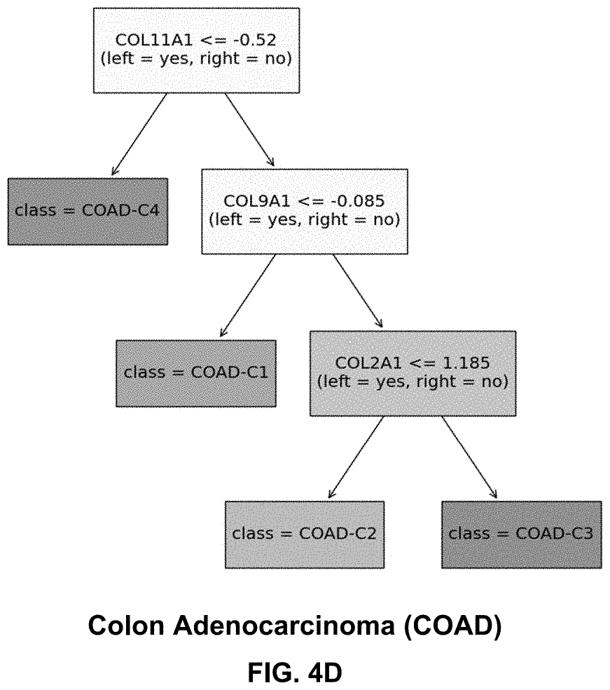

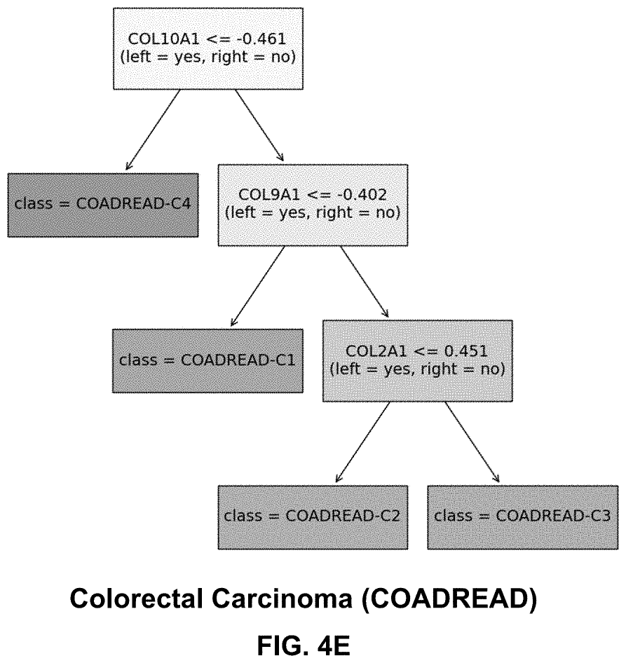

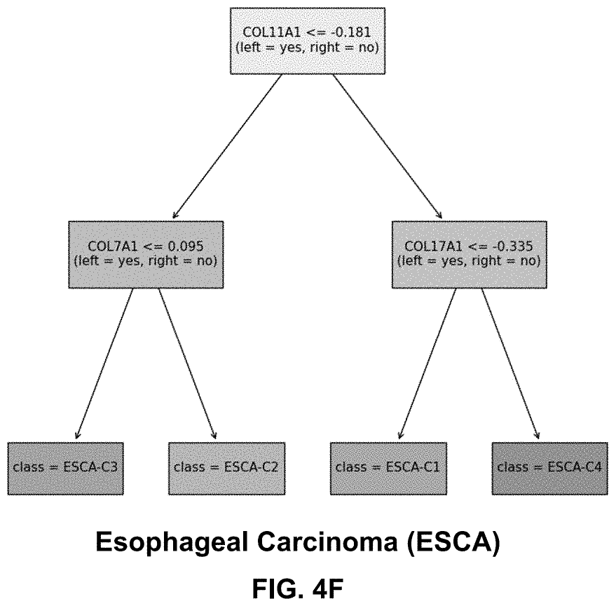

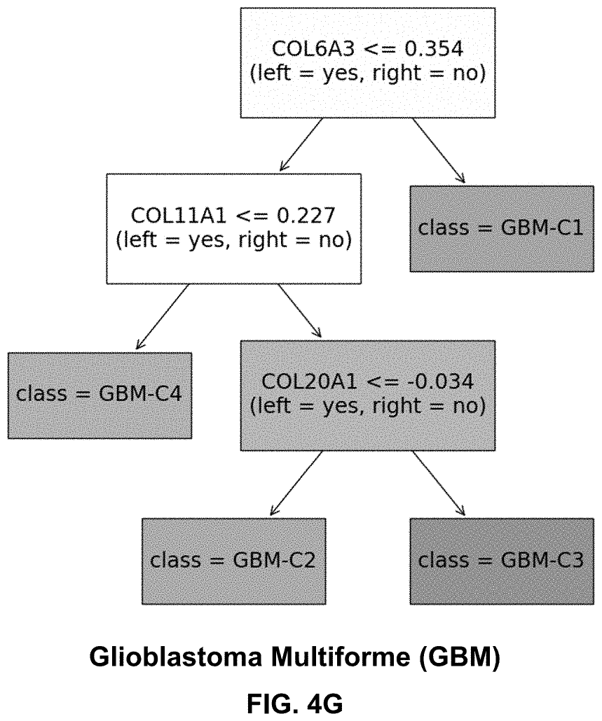

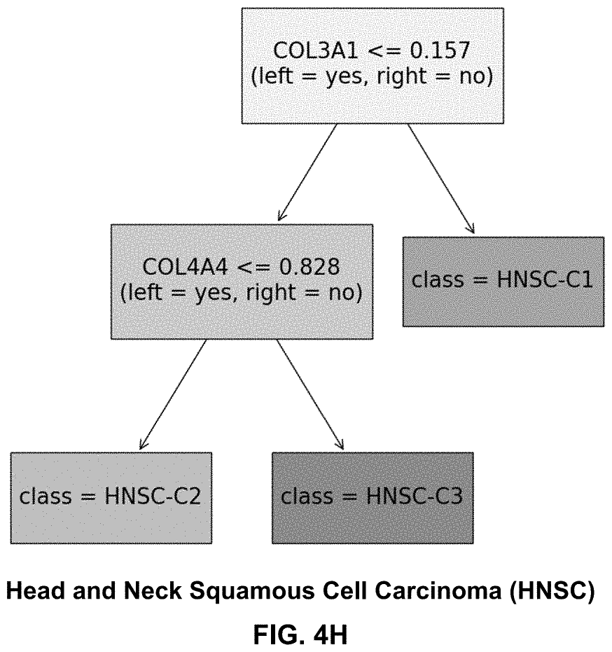

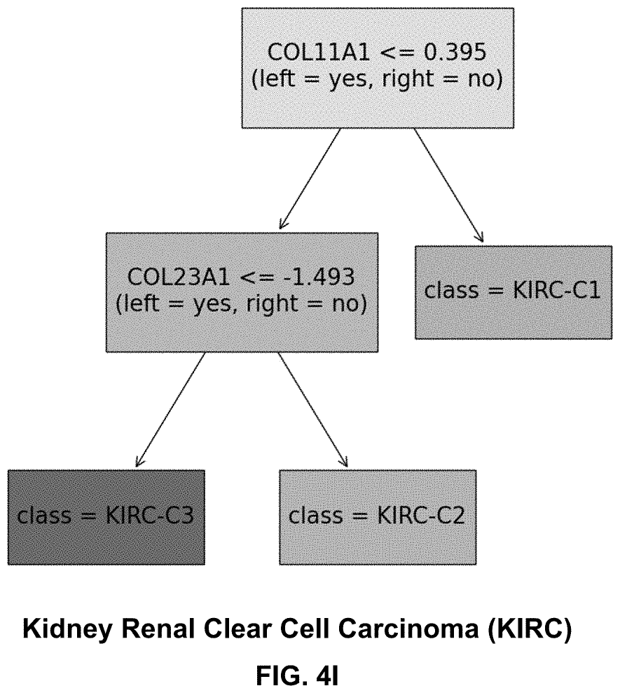

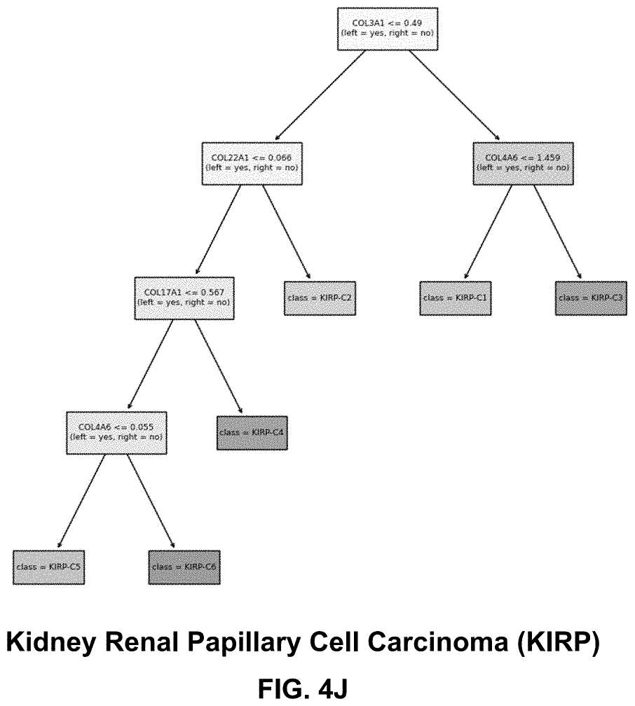

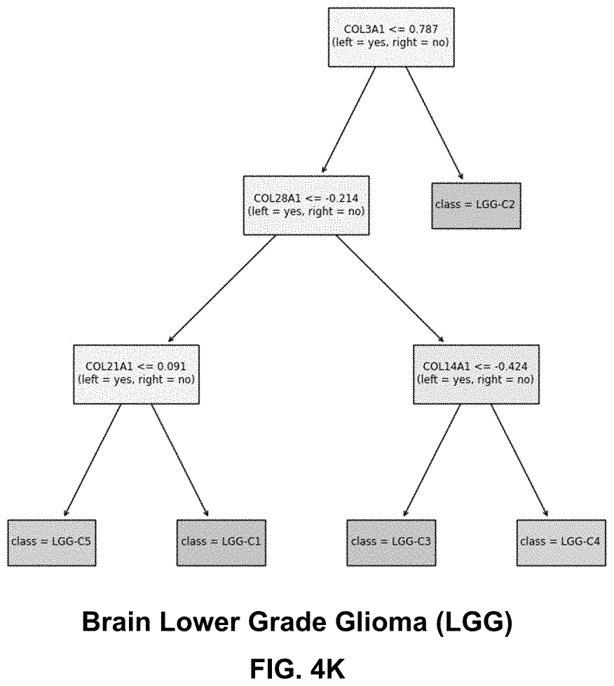

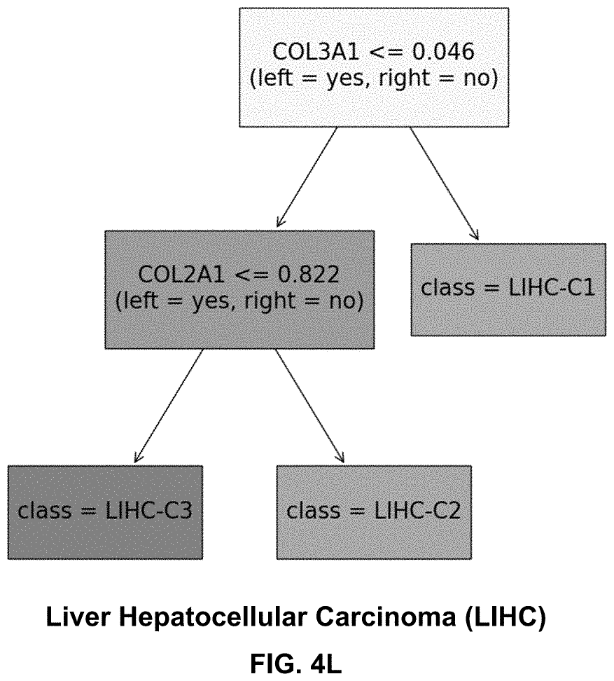

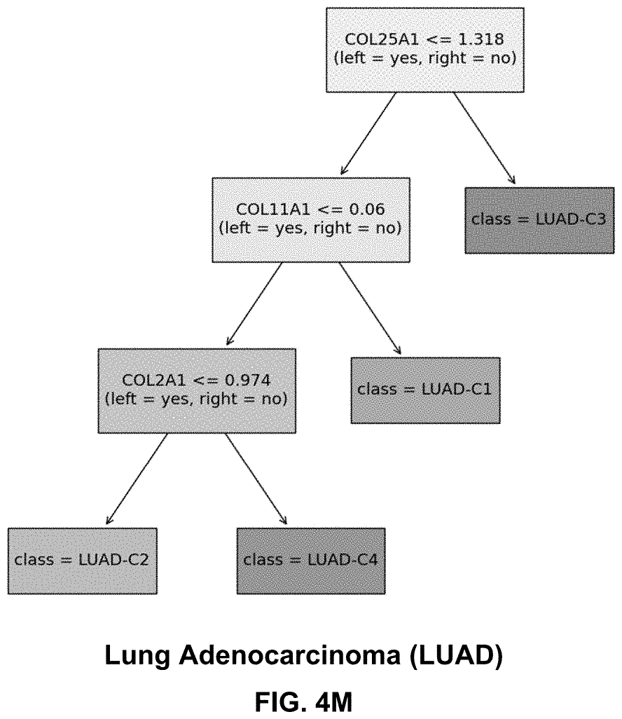

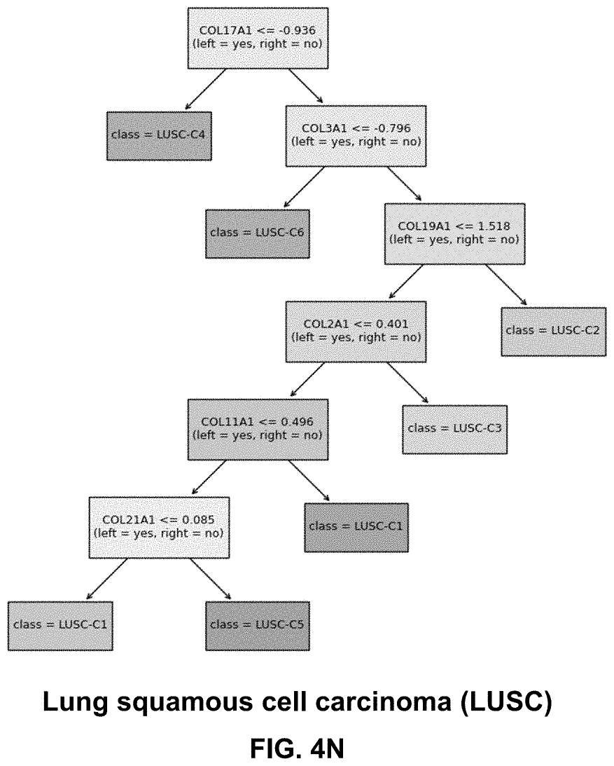

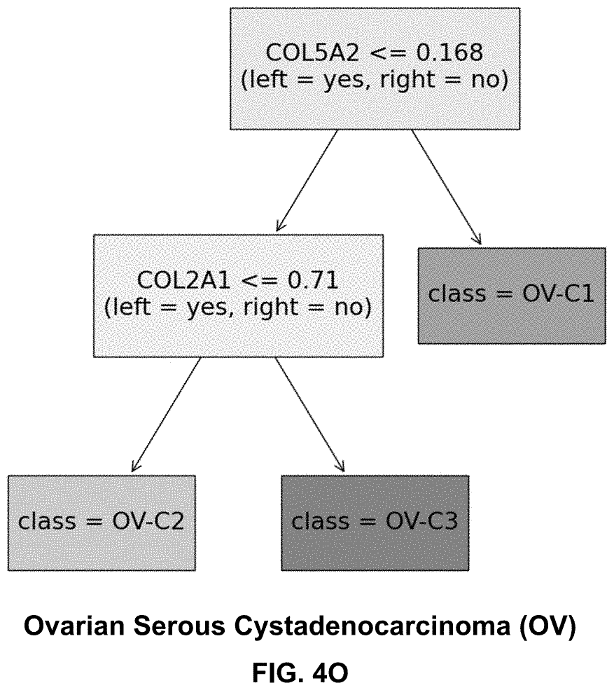

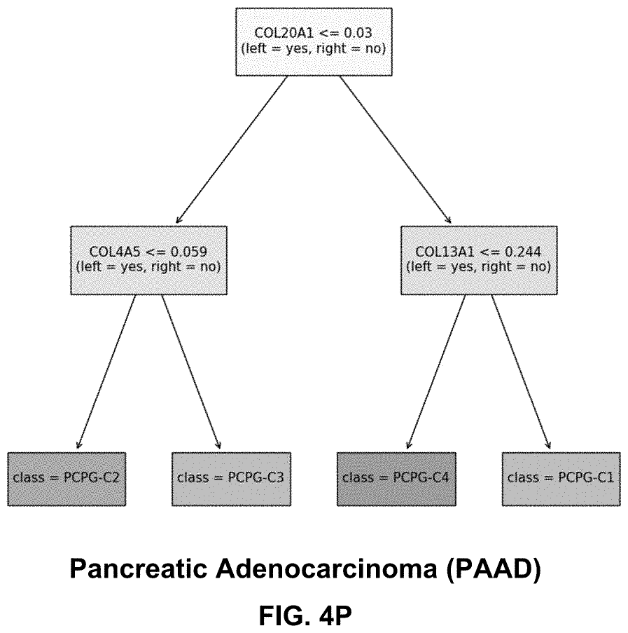

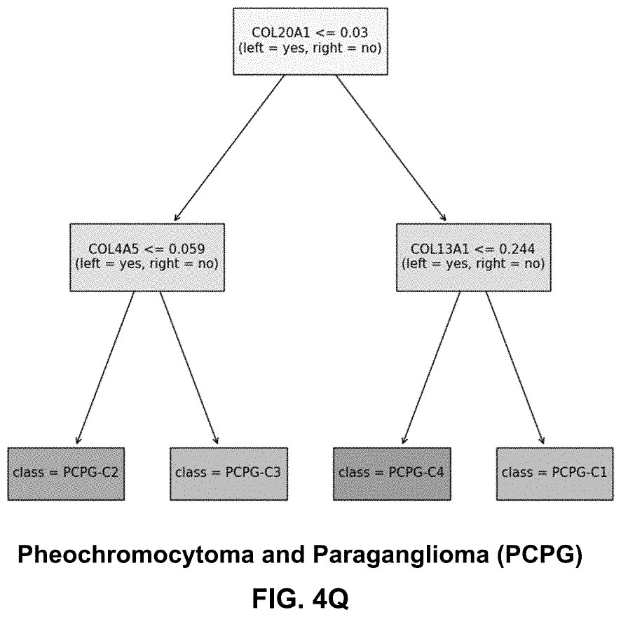

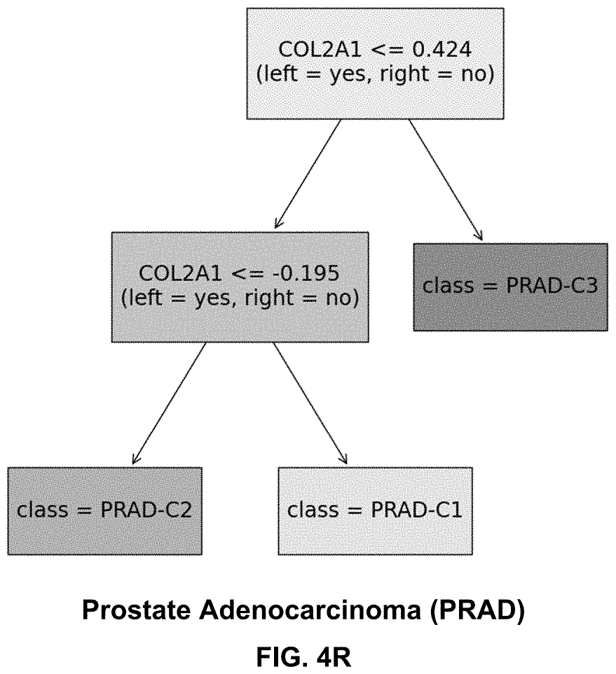

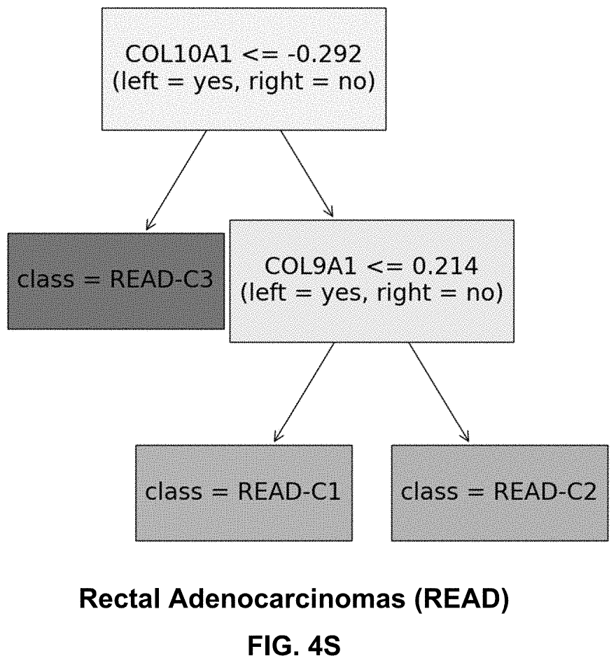

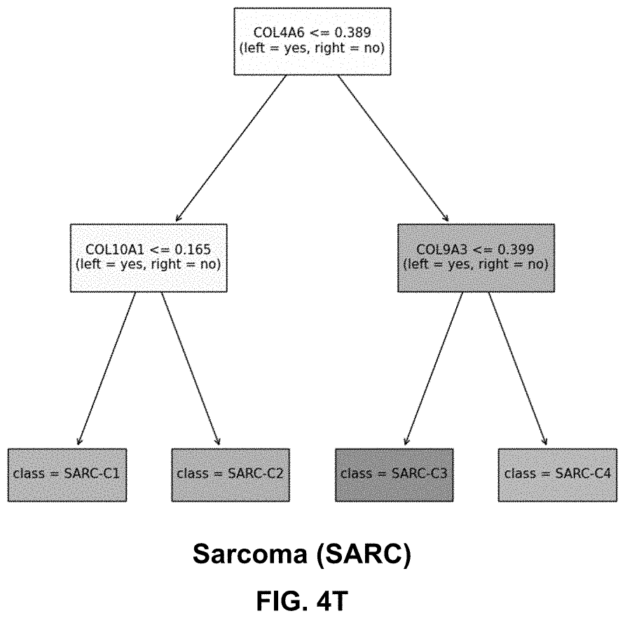

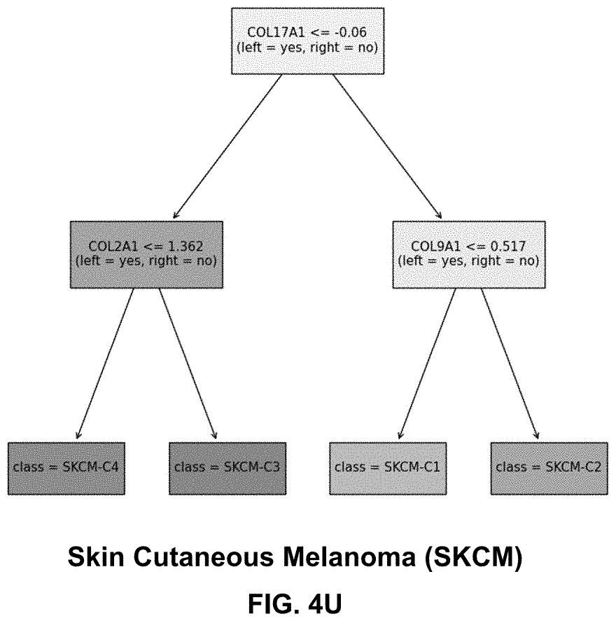

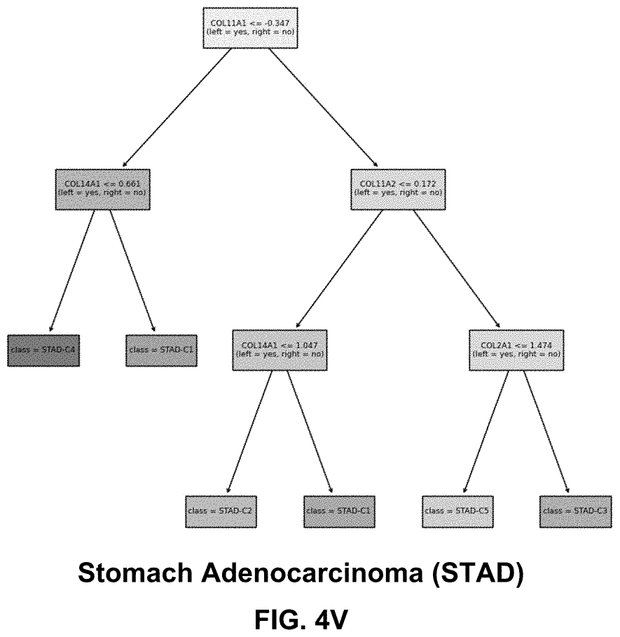

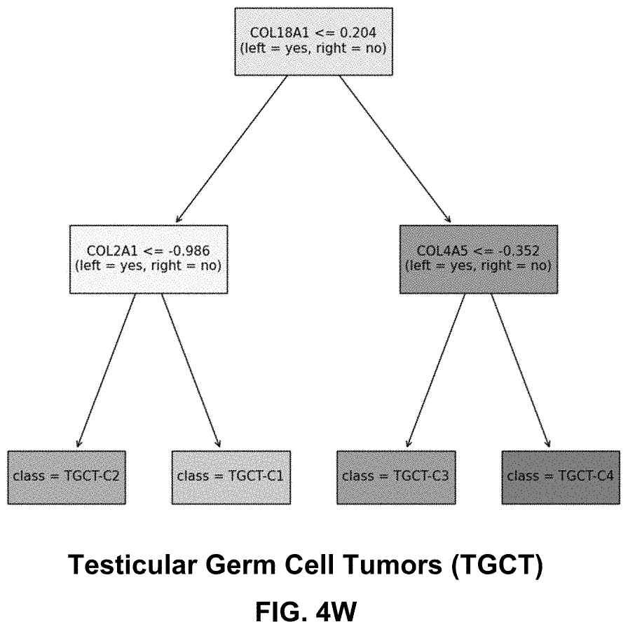

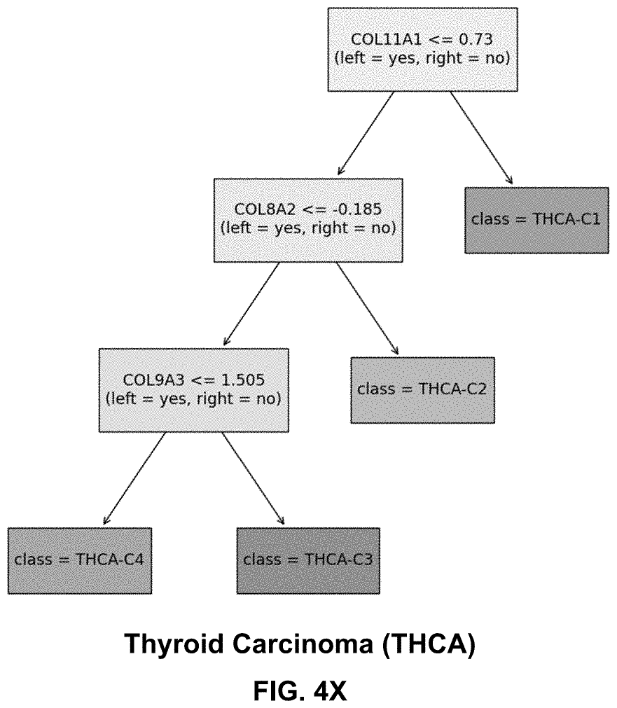

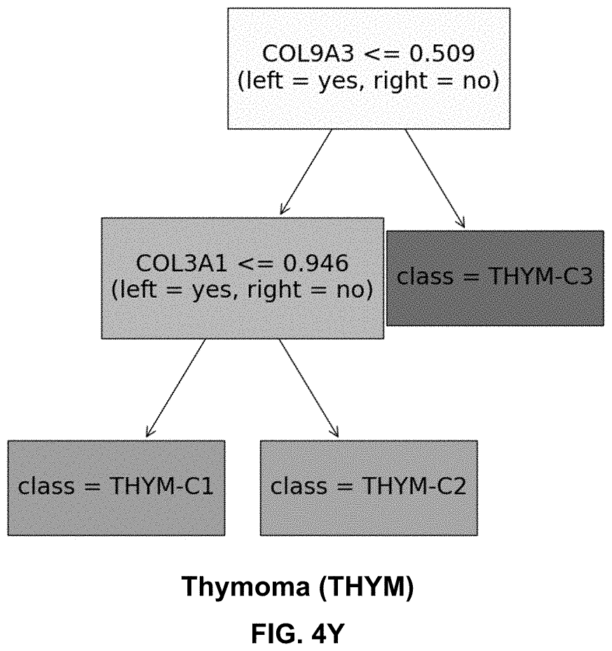

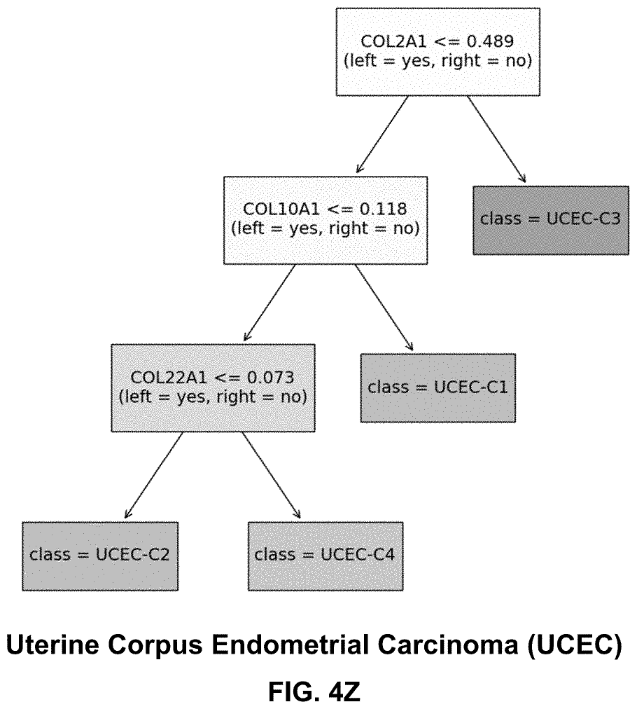

Bladder Urothelial Carcinoma (BLCA) has the biomedical art-recognized meaning. See National Cancer Institute (NCI) Dictionary of Cancer Terms. Treatments specific for bladder urothelial carcinoma are known in the biomedical art. See the decision tree in FIG. 4A.

Brain Lower Grade Glioma (LGG) has the biomedical art-recognized meaning. See National Cancer Institute (NCI) Dictionary of Cancer Terms. Treatments specific for brain lower grade glioma are known in the biomedical art. See the decision tree in FIG. 4K.

Breast Invasive Carcinoma (BRCA) has the biomedical art-recognized meaning. See National Cancer Institute (NCI) Dictionary of Cancer Terms. Treatments specific for breast invasive carcinoma are known in the biomedical art. See the decision tree in FIG. 4B.

Cancer has the biomedical art-recognized meaning. Treatments for cancer are known in the biomedical art. Cancer Cell and Tumor Cell has the biomedical art-recognized meaning of a cell undergoing early, intermediate or advanced stages of multi-step neoplastic progression as described by Pitot et al., Fundamentals of Oncology, pp. 15-28. The features of early, intermediate and advanced stages of neoplastic progression were described using microscopy. Cancer cells at each of the three stages of neoplastic progression generally have abnormal karyotypes, including translocations, inversion, deletions, isochromosomes, monosomies, and extra chromosomes.

Cyclin D1 (CCND1) has the biomedical art-recognized meaning.

CD56 has the biomedical art-recognized meaning. Neural cell adhesion molecule (NCAM), also called CD56, is a homophilic binding glycoprotein expressed on the surface of neurons, glia and skeletal muscle. Natural Killer (NK) cells are lymphocytes of the innate immune system and are important for defense against infectious pathogens and cancer. Classically, the CD56_{dim} NK cell subset is thought to mediate antitumor responses.

CDKN2A has the biomedical art-recognized meaning. CDKN2A is a tumor suppressor. CDNK2A truncations are genetic mutations in the CDNK2A.

CDKN2B has the biomedical art-recognized meaning. CDKN2V is a tumor suppressor.

Cervical Squamous Cell Carcinoma has the biomedical art-recognized meaning. Treatments specific for cervical squamous cell carcinoma are known in the biomedical art.

Cholesterol Metabolism has the biomedical art-recognized meaning.

COL10A1 has the biomedical art-recognized meaning. Collagens have been used as biomarkers for specific cell types and cell states including COL10A1 as a hypertrophic chondrocyte differentiation marker. See Shen et al., Orthodontics & Craniofacial Research (2005).

COL11A1 has the biomedical art-recognized meaning of a gene for collagen, type XI, alpha 1.

COL11A2 has the biomedical art-recognized meaning.

COL16A1 has the biomedical art-recognized meaning.

COL17A1 has the biomedical art-recognized meaning. Collagens have been used as biomarkers for specific cell types and cell states including COL17A1 marking skin stem cells. COL17A1 is also a squamous cell marker.

COL20A1 has the biomedical art-recognized meaning of the gene for collagen, type XX, alpha 1. COL20A1 is the neuronal collagen.

COL22A1 has the biomedical art-recognized meaning. Collagens have been used as biomarkers for specific cell types and cell states including COL22A1 as a chondrocyte differentiation marker. See Feng et al., (2019).

COL25A1 has the biomedical art-recognized meaning. COL25A1 is a transmembrane collagen normally expressed in brain tissue and developing myoblasts.

COL227A1 has the biomedical art-recognized meaning of the gene for collagen, type XXVII, alpha 1.

COL4A3 has the biomedical art-recognized meaning.

COL4A4 has the biomedical art-recognized meaning of the gene for collagen, type IV, alpha 4.

COL7A1 has the biomedical art-recognized meaning.

COL9A1 has the biomedical art-recognized meaning.

COL9A2 has the biomedical art-recognized meaning of the gene for collagen, type IX, alpha 2.

COL9A3 has the biomedical art-recognized meaning of the gene for collagen, type IX, alpha 3.

Collagen RNA Expression Groups (ColClusters) have the meaning described in this specification. ColClusters are defined through unsupervised k-means clustering in each The Cancer Genome Atlas cancer type and across 8,646 solid The Cancer Genome Atlas tumors into fifteen groups. These classifications clusters were defined by collagen composition enriched in tissue specificity, cell states, immune environment, molecular alterations and overall survival.

Collagen has the biomedical art-recognized meaning. Collagens constitute the major component of the tumor extracellular matrix but have been mostly overlooked as simple structural proteins. Collagens do far more than just form structures. The function of the full panoply of forty-three collagen genes in tumors remains underappreciated. Collagens are a large complex family of protein with a wide range of structures and tissue specific expression. Minor collagens are informally defined as any collagen at lower expression levels compared to the major structural collagens (types I, II, and III) found in high abundance in many tissues. Fibrillar collagens constitute a subgroup of collagens and include type I and many collagens that interact with collagen type I including collagen types V, XI, XII, XIV, and XVII. See Ricard-Blum, Cold Spring Harbor Perspectives in Biology (2011).

TABLE 1
Collagens

| Structural | | |
|---|---|---|
| Family | Gene | Putative Role |
| Fibril forming | COL1A1 | Fiber collagen |
| | COL1A2 | Fiber collagen |
| | COL2A1 | Fiber collagen |
| | COL3A1 | Fiber collagen |
| | COL5A1; | Promotes Type I fibers |
| | COL5A2 | |
| | COL5A3 | Negative regulator of Type I fibers |
| | COL11A1 | Promotes Type I fibers |
| | COL11A2 | Promotes Type I fibers |
| | COL14A1 | Fibril surface; Negative regulator of |
| | COL24A1; | Type I fibers |
| | COL27A1 | Type I fibrilogenesis regulator |
| FACIT | COL9A1 | |
| | COL9A2 | |
| | COL9A3 | |
| | COL12A1 | |
| Network | COL15A1 | Banded Fibril linker Basement membrane |
| | COL19A1 | zones |
| | COL20A1 | |
| | COL21A1 | |
| | COL22A1 | |
| | COL4A1 | Basement membrane zones Basement |
| | COL4A2 | Basement |
| | COL8A1 | Basement |
| | COL10A1 | Chondrocyte matrix deposition |
| COL6 | COL6A1 | Basement membrane/interstitial matrix |
| | COL6A2, | Basement membrane/interstitial matrix |
| | COL6A3 | |
| Membrane | COL7A1; | Dermoepidermal Anchoring fibril |
| | COL26A1; | |
| | COL28A1 | |
| | COL13A1 | |
| | COL17A1; | Dermoepidermal anchoring complex Not |
| | COL23A1 | known function |
| | COL25A1 | Linked with amyloid formation |
| Multiplexins | COL18A1 | |

Collagens are one family of proteins that constitute the matrisome. Several groups have investigated classifications defined by large sets of matrisome. Izzi et al., Matrix Biology Plus (2019).

Post-translational modifications of collagens and remodeling of the matrix, often by proteolytic cutting of collagens, spatial location of collages within the tumor are features of collagen complexity.

Collagen type I has the biomedical art-recognized meaning. Type I collagen is the most abundant collagen of the human body. It forms large, eosinophilic fibers known as collagen fibers. It is present in scar tissue, the end product when tissue heals by repair, and tendons, ligaments, the endomysium of myofibrils, the organic part of bone, the dermis, the dentin, and organ capsules. The COL1A1 gene produces the pro-alpha1(I) chain. This chain combines with another pro-alpha1(I) chain and with a pro-alpha2(I) chain (produced by the COL1A2 gene) to make a molecule of type I procollagen. These triple-stranded, rope-like procollagen molecules is processed by enzymes outside the cell. After these molecules are processed, they arrange themselves into long, thin fibrils that cross-link to one another in the spaces around cells. The cross-links result in the formation of very strong mature type I collagen fibers.

Colon Adenocarcinoma (COAD) has the biomedical art-recognized meaning. See National Cancer Institute (NCI) Dictionary of Cancer Terms. Treatments specific for colon adenocarcinoma are known in the biomedical art. See the decision tree in FIG. 4D.

Colorectal Carcinoma (COADREAD) has the biomedical art-recognized meaning. See National Cancer Institute (NCI) Dictionary of Cancer Terms. Treatments specific for colorectal carcinoma are known in the biomedical art. See the decision tree in FIG. 4E.

Combination Therapy has the oncological art-recognized meaning of administration of each agent or therapy in a sequential manner in a regimen that will provide beneficial effects of the combination, and co-administration of these agents or therapies in a substantially simultaneous manner, such as in a single capsule having a fixed ratio of these active agents or in multiple, separate capsules for each agent. Combination therapy also includes combinations where individual elements may be administered at different times and/or by different routes but which act in combination to provide a beneficial effect by co-action or pharmacokinetic and pharmacodynamics effect of each agent or tumor treatment approaches of the combination therapy.

Comprises and comprising refer to elements, components, or steps in a non-exclusive manner, indicating that the referenced elements, components, or steps may be present, used, or combined with other elements, components, or steps. The singular terms “a,” “an,” and “the” include plural referents unless context indicates otherwise. Similarly, the inclusive term “or” should cover the term “and” unless the context indicates otherwise. The abbreviation “e.g.” means a non-limiting example and is synonymous with the term “for example.”

Copy number alteration (CNA) has the biomedical art-recognized meaning.

Cytotoxic cell has the biomedical art-recognized meaning.

Dendritic cell has the biomedical art-recognized meaning.

DK6 has the biomedical art-recognized meaning.

DNA Repair has the biomedical art-recognized meaning.

E2F has the biomedical art-recognized meaning.

Effector Memory T (Tₑₘ) cells has the biomedical art-recognized meaning.

EGFR has the biomedical art-recognized meaning.

Epithelial Mesenchymal Transition (EMT) has the biomedical art-recognized meaning.

Endocervical Adenocarcinoma (CESC) has the biomedical art-recognized meaning. See National Cancer Institute (NCI) Dictionary of Cancer Terms. Treatments specific for endocervical adenocarcinoma are known in the biomedical art. See the decision tree in FIG. 4C.

Eosinophil has the biomedical art-recognized meaning.

Estrogen receptor positive (ER⁺) has the biomedical art-recognized meaning of cells with a protein that binds to the hormone estrogen. Cancer cells that are estrogen receptor positive may need estrogen to grow. These cells may stop growing or die when treated with substances that block the binding and actions of estrogen.

Esophageal Carcinoma (ESCA) has the biomedical art-recognized meaning. See National Cancer Institute (NCI) Dictionary of Cancer Terms. Treatments specific for esophageal carcinoma are known in the biomedical art. See the decision tree in FIG. 4F.

Extra cellular matrix (ECM) has the biomedical art-recognized meaning. The extracellular matrix is a critical determinant of tumor fate that reflects the output from myriad cell types in the tumor. The impact of the composition of the extracellular matrix on patient outcomes remains largely unknown.

FAT1 has the biomedical art-recognized meaning. FAT1 truncations.

Fatty Acid Metabolism has the biomedical art-recognized meaning.

Fibroblast Growth Factor (FGF) has the biomedical art-recognized meaning. The FGF locus is the human chromosome location of the FGF gene.

Fibroblast Growth Factor 3 (FGFR3) has the biomedical art-recognized meaning.

G2M Checkpoint has the biomedical art-recognized meaning. The G2-M DNA damage checkpoint is an important cell cycle checkpoint in eukaryotic organisms that ensures that cells don't initiate mitosis until damaged or incompletely replicated DNA is sufficiently repaired. Cells which have a defective G2-M checkpoint, if they enter M phase before repairing their DNA, it leads to apoptosis or death after cell division.

Gamma Delta T cells has the biomedical art-recognized meaning.

Glioblastoma Multiforme (GBM) has the biomedical art-recognized meaning of a fast-growing glioma that develops from star-shaped glial cells (astrocytes and oligodendrocytes) that support the health of the nerve cells within the brain. Glioblastoma multiforme is often called a grade IV astrocytoma. See National Cancer Institute (NCI) Dictionary of Cancer Terms. Treatments specific for Glioblastoma multiforme are known in the biomedical art. See the decision tree in FIG. 4G.

Glycolysis has the biomedical art-recognized meaning.

Hallmark Gene Sets has the biomedical art-recognized meaning. In this specification, the collagen defined tumor groups (classifiers) have distinct phenotypes i.e., hallmark gene sets, and distinct immunoenvironments, thus providing ways to target these groups of tumors.

Head and Neck Squamous Cell Carcinoma (HNSC) has the biomedical art-recognized meaning. See National Cancer Institute (NCI) Dictionary of Cancer Terms. Treatments specific for head and neck squamous cell carcinoma are known in the biomedical art. See the decision tree in FIG. 4H.

Hedgehog Signaling has the biomedical art-recognized meaning.

HRAS has the biomedical art-recognized meaning.

Human Chromosome has the biomedical art-recognized meaning.

Hypoxia has the biomedical art-recognized meaning.

iDC cells has the biomedical art-recognized meaning.

IDH1 has the biomedical art-recognized meaning.

IFNγ has the biomedical art-recognized meaning.

IL2 Stat5 Signaling has the biomedical art-recognized meaning.

Immunocompetent environment has the biomedical art-recognized meaning.

Inflammatory has the biomedical art-recognized meaning.

Inflammatory Response has the biomedical art-recognized meaning.

Interferon Gamma Response has the biomedical art-recognized meaning.

Kidney Renal Clear Cell Carcinoma (KIRC) has the biomedical art-recognized meaning. See National Cancer Institute (NCI) Dictionary of Cancer Terms. Treatments specific for kidney renal clear cell carcinoma are known in the biomedical art. See the decision tree in FIG. 4I.

Kidney Renal Papillary Cell Carcinoma (KIRP) has the biomedical art-recognized meaning. See National Cancer Institute (NCI) Dictionary of Cancer Terms. Treatments specific for kidney renal papillary cell carcinoma are known in the biomedical art. See the decision tree in FIG. 4J.

KMT2C has the biomedical art-recognized meaning.

KMT2D has the biomedical art-recognized meaning.

KRAS has the biomedical art-recognized meaning. KRAS is an oncogene.

Liver Hepatocellular Carcinoma (LIHC) has the biomedical art-recognized meaning. Treatments specific for liver hepatocellular carcinoma are known in the biomedical art. See the decision tree in FIG. 4L.

LRP1B has the biomedical art-recognized meaning.

Lung Adenocarcinoma (LUAD) has the biomedical art-recognized meaning. Treatments specific for lung adenocarcinoma are known in the biomedical art. See the decision tree in FIG. 4M.

Lung Squamous Cell Carcinoma (LUSC) has the biomedical art-recognized meaning. See National Cancer Institute (NCI) Dictionary of Cancer Terms. Treatments specific for lung squamous cell carcinoma are known in the biomedical art.

Lymphocyte Depleted has the biomedical art-recognized meaning.

Macrophages has the biomedical art-recognized meaning.

Mast Cell has the biomedical art-recognized meaning.

Met has the biomedical art-recognized meaning.

Microsatellite instability (MSI) has the biomedical art-recognized meaning.

Missense has the biomedical art-recognized meaning. Missense is a genetic mutation.

MSS Tumor has the biomedical art-recognized meaning of cancer cells that are microsatellite stable. See National Cancer Institute (NCI) Dictionary of Cancer Terms. MSS Tumors have been called “cold” tumors.

MSIH Tumor has the biomedical art-recognized meaning of cancer cells with a high number of mutations (changes) within microsatellites. See National Cancer Institute (NCI) Dictionary of Cancer Terms. Knowing whether cancer is microsatellite instability-high may help plan the best treatment. Also called microsatellite instability-high cancer.

MTAP has the biomedical art-recognized meaning. MTAP is a tumor suppressor.

mTORC Signaling has the biomedical art-recognized meaning.

mTORC1 Signaling has the biomedical art-recognized meaning.

Multi-omics, integrative omics, ‘panomics’ or ‘pan-omics’ is a biological analysis approach in which the data sets are multiple ‘omes,’ such as the genome, proteome, transcriptome, epigenome, metabolome, and microbiome (i.e., a meta-genome or meta-transcriptome, depending upon how it is sequenced). See Bersanelli et al., Methods for integrating multi-omics data: mathematical aspects. BMC Bioinformatics. 17 (2): S15 (Jan. 1, 2016); Bock et al., Multi-Omics of Single Cells: Strategies and Applications. Trends in Biotechnology. 34 (8): 605-608 (August 2016); and Vilanova & Porcar, Are multi-omics enough? Nature Microbiology. 1(8): 16101 (Jul. 26, 2016).

MYC has the biomedical art-recognized meaning. MYC is an oncogene.

Myc Targets has the biomedical art-recognized meaning.

Myogenesis has the biomedical art-recognized meaning.

Neutrophil has the biomedical art-recognized meaning.

NF1 has the biomedical art-recognized meaning. NF1 truncations

Notch has the biomedical art-recognized meaning. Notch can be a tumor suppressor pathway.

Ovarian Serous Cystadenocarcinoma (OV) has the biomedical art-recognized meaning. See National Cancer Institute (NCI) Dictionary of Cancer Terms. Ovarian serous cystadenocarcinoma is a copy number driven cancer with low mutation rates. Ovarian serous cystadenocarcinoma is a copy number driven cancer with low mutation rates. Treatments specific for ovarian serous cystadenocarcinoma are known in the biomedical art. See the decision tree in FIG. 40.

Oxidative Phosphorylation has the biomedical art-recognized meaning.

P53 has the biomedical art-recognized meaning. P53 is a tumor suppressor. TP53, a caretaker gene, encodes the protein p53, which is nicknamed “the guardian of the genome”. p53 has many functions in the cell including DNA repair, inducing apoptosis, transcription, and regulating the cell cycle.

PanCan collagen clustering links collagen expression and classification to tissue specificity and lineages.

PanCancer means across all twenty-six cancer types in the TCGA dataset. It means that the collagens identified specific groupings across cancers. Collagen clusters were specific for certain tumor types. Other collagen clusters brought together cancer types with similar molecular features. PanCancer clustering can similar collagen and ECM environments across tumor types that could be targeted.

Pancreatic Adenocarcinoma (PAAD) has the biomedical art-recognized meaning. See National Cancer Institute (NCI) Dictionary of Cancer Terms. Treatments specific for pancreatic adenocarcinoma are known in the biomedical art. See the decision tree in FIG. 4P.

Pheochromocytoma and Paraganglioma (PCPG) has the biomedical art-recognized meaning. See National Cancer Institute (NCI) Dictionary of Cancer Terms. Treatments specific for pancreatic adenocarcinoma are known in the biomedical art. See the decision tree in FIG. 4Q.

PI3K AKT MTOR Signaling has the biomedical art-recognized meaning.

PIK3CA has the biomedical art-recognized meaning.

Prostate Adenocarcinoma (PRAD) has the biomedical art-recognized meaning. See National Cancer Institute (NCI) Dictionary of Cancer Terms. Treatments specific for prostate adenocarcinoma are known in the biomedical art. See the decision tree in FIG. 4R.

Pattern 1, where specific molecular alterations were localized to one or two ColClusters, has the meaning described in this specification.

Pattern 2, where similar molecular alterations have distinct tumor extracellular matrix composition, has the meaning described in this specification.

Protein Secretion has the biomedical art-recognized meaning.

PTEN has the biomedical art-recognized meaning.

Quantitative Set Analysis for Gene Expression (QuSAGE) has the biomedical art-recognized meaning. See Meng et al., PLoS Computational Biology, 15(4), e1006899 (2019).

RAD21 has the biomedical art-recognized meaning.

RB1 has the biomedical art-recognized meaning. RB1 is a tumor suppressor. RB1 is reported to link the cell cycle, adhesion and the tumor environment Engel et al., (2014).

Reactive Oxygen Species has the biomedical art-recognized meaning.

Rectum Adenocarcinoma (READ) has the biomedical art-recognized meaning. See National Cancer Institute (NCI) Dictionary of Cancer Terms. Treatments specific for rectum adenocarcinoma are known in the biomedical art. See the decision tree in FIG. 4S.

Regulatory T cell (Treg) has the biomedical art-recognized meaning.

Sarcoma (SARC) has the biomedical art-recognized meaning. See National Cancer Institute (NCI) Dictionary of Cancer Terms. Treatments specific for Prostate Adenocarcinoma are known in the biomedical art. See the decision tree in FIG. 4T.

Skin Cutaneous Melanoma (SKCM) has the biomedical art-recognized meaning. See National Cancer Institute (NCI) Dictionary of Cancer Terms. Treatments specific for skin cutaneous melanoma are known in the biomedical art. See the decision tree in FIG. 4U.

SOX2 has the biomedical art-recognized meaning.

Stomach Adenocarcinoma (STAD) has the biomedical art-recognized meaning. See National Cancer Institute (NCI) Dictionary of Cancer Terms. Treatments specific for stomach adenocarcinoma are known in the biomedical art. See the decision tree in FIG. 4V.

Subject has the plain meaning of an individual, e.g., a vertebrate, e.g., a mammal, e.g., a human or patient to be tested or treated by the method of the invention.

Support Vector Machine (SVM) has the biomedical art-recognized meaning of a method that predicts tumors with high aneuploidy based on collagen expression patterns.

T helper cell has the biomedical art-recognized meaning.

The Cancer Genome Atlas (TCGA) has the biomedical art-recognized meaning.

TERC has the biomedical art-recognized meaning.

Testicular Germ Cell Tumors (TGCT) has the biomedical art-recognized meaning. See National Cancer Institute (NCI) Dictionary of Cancer Terms. Treatments specific for testicular germ cell tumors are known in the biomedical art. See the decision tree in FIG. 4W.

TGFβ has the biomedical art-recognized meaning.

Thymoma (THYM) has the biomedical art-recognized meaning. See National Cancer Institute (NCI) Dictionary of Cancer Terms. Treatments specific for thyoma are known in the biomedical art. See the decision tree in FIG. 4Y.

Thyroid Carcinoma (THCA) has the biomedical art-recognized meaning. See National Cancer Institute (NCI) Dictionary of Cancer Terms. Treatments specific for thyroid carcinoma are known in the biomedical art. See the decision tree in FIG. 4X.

Tumor Microenvironment (TME) has the biomedical art-recognized meaning.

TP53 has the biomedical art-recognized meaning. TP53, a caretaker gene, encodes the protein p53, which is nicknamed “the guardian of the genome”. p53 has many functions in the cell including DNA repair, inducing apoptosis, transcription, and regulating the cell cycle. TP53 is the most frequently mutated gene and has been linked to remodeling the extracellular matrix. See Kastenhuber & Lowe (2017).

Pharmaceutically acceptable salts include but are not limited to salts of acidic or basic groups. Basic compounds can form a wide variety of salts with various inorganic and organic acids. Compounds that include an amino moiety can form pharmaceutically acceptable salts with various amino acids. Acidic compounds can form base salts with different pharmacologically acceptable cations. Salts include quaternary ammonium salts of the compounds described, where the compounds have one or more tertiary amine moiety.

Pharmaceutically acceptable has the biomedical art-recognized meaning that the compounds, materials, compositions, or dosage forms are within the scope of sound medical judgment and are suitable for contact with tissues of humans and other animals. The pharmaceutically acceptable compounds, materials, compositions, or dosage forms result in no persistent detrimental effect on the subject or the general health of the treated subject. Still, transient effects, such as minor irritation or a stinging sensation, are common with the administration of medicament and follow the composition, formulation, or ingredient, e.g., excipient, in question. Guidance about what is pharmaceutically acceptable is provided by comparable compounds, materials, compositions, or dosage forms in the US Pharmacopeia or another generally recognized pharmacopeia for use in animals, particularly in humans.

Therapeutically Effective amount has the biomedical art-recognized meaning of the amount of active compound or pharmaceutical agent that elicits the biological or medicinal response sought in a tissue, system, animal, individual, or human by a researcher, veterinarian, medical doctor, or another clinician. The therapeutic effect depends upon the disorder being treated or the biological effect desired. The therapeutic effect can be a decrease in the severity of symptoms associated with the disorder or inhibition (partial or complete) of progression of the disorder, or improved treatment, healing, prevention or elimination of a disorder, or side-effects. The amount needed to elicit the therapeutic response can be based on, for example, the age, health, size, and sex of the subject. Optimal amounts can also be determined based on monitoring of the response to treatment.

Treatment, Treat, or Treating has the biomedical art-recognized meaning that includes any treatment of a disease or condition of a mammal, for example, a human, and includes, without limitation: (a) preventing the disease or condition from in a subject which may be predisposed to the disease or condition; (b) inhibiting the disease or condition, i.e., arresting its development; (c) relieving and or ameliorating the disease or condition, i.e., regressing the disease or condition; or (d) curing the disease or condition, i.e., stopping its development or progression. The population of subjects treated by the methods of the invention includes subjects suffering from the undesirable condition or disease and subjects at risk for development of the condition or disease.

Truncation has the biomedical art-recognized meaning. Truncation is a genetic mutation.

Tumor has the biomedical art-recognized meaning.

Tumor Classification Associated with High and Low Overall Survival has the meaning described in this specification.

Tumor Microenvironment has the biomedical art-recognized meaning.

Unfolded Protein Response has the biomedical art-recognized meaning.

Uterine Corpus Endometrial Carcinoma (UCEC) has the biomedical art-recognized meaning. See National Cancer Institute (NCI) Dictionary of Cancer Terms. Treatments specific for uterine corpus endometrial carcinoma are known in the biomedical art. See the decision tree in FIG. 4Z.

Wnt Beta Catenin Signaling has the biomedical art-recognized meaning. See Pai et al., Journal of Hematology & Oncology, 10, 101 (2017).

Wnt Signaling has the biomedical art-recognized meaning. See Komiya & Habas, Wnt signal transduction pathways. Organogenesis, 4(2):68-75 (April 2008).

Wound Healing has the biomedical art-recognized meaning.

Xenobiotic Metabolism has the biomedical art-recognized meaning.

Unless otherwise defined, scientific and technical terms used with this application shall have the meanings commonly understood by persons having ordinary skill in the biomedical art. This invention is not limited to the particular methodology, protocols, reagents, etc., described herein and can vary.

The disclosure described herein does not concern a process for cloning humans, methods for modifying the germ line genetic identity of humans, uses of human embryos for industrial or commercial purposes, or procedures for modifying the genetic identity of animals likely to cause them suffering with no substantial medical benefit to man or animal and animals resulting from such processes.

Guidance from Materials and Methods

A person having ordinary skill in the art can use these materials and methods as guidance to predictable results when making and using the invention:

k-means clustering with gap statistical analysis. k-means clustering is a method of vector quantization, originally from signal processing, that aims to partition n observations into k clusters in which each observation belongs to the cluster with the nearest mean (cluster centers or cluster centroid), serving as a prototype of the cluster.

Association/enrichment with histology, clinicopathological characteristics including patient outcomes and overall survival.

Association/enrichment assessment of mutations and copy number alterations.

ssGSEA for pathway and immune signature assessment with Kolmogorov-Smirnov tests. Single-sample GSEA (ssGSEA), an extension of Gene Set Enrichment Analysis (GSEA), calculates separate enrichment scores for each pairing of a sample and gene set. Each ssGSEA enrichment score represents how much the genes in a particular gene set are coordinately up- or down-regulated within a sample.

Statistical Analysis and Visualization. Kaplan-Meier and Cox survival analysis, based on clinical data from the PanCancer Atlas were used to compare overall survival between clusters, using the Python lifelines and R survival packages respectively. The log-rank test was performed on the resulting Kaplan-Meier survival models to assess differences in overall survival within tumors. Categorical variables were compared with Pearson's Chi-squared test. Unless otherwise stated, all comparisons for continuous variables were performed with the Kolmogorov-Smirnov test. Where applicable, *, **, *** denote p values of less than 0.05, 0.01, and 0.001 respectively. Graphs and heatmaps were generated using the Seaborn data visualization library for Python.

Deposited data can be discovered by persons having ordinary skill in the biomedical art. The NCI Genome Data Commons (https://gdc.cancer.go) contains processed clinical and sequence data. Thorsson et al., Immunity (2018) provides aneuploidy score, stromal fraction, and mutation rate data.

Aneuploidy scores, stromal fractions, and overall mutation rates were taken from Thorsson et al., Immunity (2018). Unless otherwise noted, all other data (clinical data, RNAseq scores, ploidy, copy number annotations, etc.), was retrieved from the respective PanCancer Atlas datasets on cBioPortal.

Clustering was based upon RNA-Seq expression data. Only primary solid tumors were considered in this analysis. From a total set of forty-three collagen genes, genes with significant expression (defined to be greater than ten samples with a RSEM expression value of 200 or greater) were selected as features for clustering. Expression values were log 2-transformed, and cancer cases were subtyped using k-means clustering with Pearson's correlation distance, for three-six clusters. Cluster number selection was informed by silhouette analysis and gap statistic comparison colon adenocarcinomas (COAD) and rectal adenocarcinomas (READ) were clustered both separately and together as a combined colorectal adenocarcinoma tumor type (COADREAD).

To characterize the molecular-level characteristics of each cluster, genesets were selected from the Molecular Signatures Database (MSigDB), and clusters were compared to each other using quantitative set analysis for gene expression (QuSAGE). This analysis was supplemented with single-sample gene set enrichment analysis (ssGSEA).

To assess the relationship between collagen expression and aneuploidy, we trained a linear support vector machine model for each tumor type with the scikit-learn machine learning package for Python. Normalized collagen RSEM expression scores and stromal fraction were used as initial input features. Feature selection was performed by removing insignificantly expressed collagens and lesser contributing (as defined by low relative Support Vector Machine weight) collagens. Labels (high and low) for each sample were generated by fitting each aneuploidy score distribution to a mixture of two Gaussian distributions. five-fold cross validated models were evaluated with area under the receiver-operator curve (AUROC) scores. The same pipeline was used to separately predict chromosome arm copy number gains and losses for copy number modifications with sufficient counts (ten copy number modifications within the tumor type).

The following EXAMPLES are provided to illustrate the invention and shall not limit the scope of the invention.

### Example 1

Expression of Collagens as Prognostic Markers in Cancer. Classification of Tumors by Collagen Expression Reveals Genotype-Tumor Extracellular Matrix Interactions.

The goal of this EXAMPLE is to classify tumors by their microenvironment properties and connect specific microenvironments with somatic mutations and copy number alterations. The inventors used k-means clustering to classify tumors using the forty-three collagen genes with gap statistics to determine the appropriate number of clusters. The inventors clustered PanCancer, across all the solid tumors with greater than 100 samples. The inventors also clustered each tumor type separately. In PanCancer, collagen clustered tumors to the tissues of origins. In specific tumors, collagen clusters were associated with specific somatic mutation and copy number alteration patterns and overall survival.

After clustering, the inventors evaluated immune cell signatures and cancer hallmarks in the clusters by ssGSEA. The inventors determined significance with a Kolmogorov-Smirnov test of the ssGSEA enrichment scores.

Using the dataset, the mRNA expression of the forty-three collagen genes classifies tumors by their cell of origin, similar to published reports. K-means clustering in each tumor type by collagen mRNA expression revealed classifications strongly associated with overall survival, specific pathways, and immune cell signatures. The collagen-defined groups were strongly associated with specific somatic mutations, copy number changes, ploidy, and aneuploidy levels. The collagen clusters also revealed specific immunoenvironments, showing which tumors were most likely to respond to immunotherapy. To further evaluate these enrichments, the inventors developed a machine learning model to predict which tumors have high or low aneuploidy and specific gain or loss of chromosome arms based on collagen expression, highlighting the connection between collagen expression and specific cancer genomes with areas under the curve above 0.8 for many tumor types including all the GI tumors. Clusters with high total collagen were typically associated with lower aneuploidy levels, and tumors with high aneuploidy were grouped in clusters with a mix of minor collagens and lower collagen type I.

Steps in the Method for Classifying by Collagen mRNA Expression

Select only solid tumors with >100 cases, 9,029 tumors.

RNAseq RSEM Scores Normalized, batch corrected.

K-means clustering.

Silhouette analysis and Gap Statistic to determine the number of clusters.

Conclusions

Collagen mRNA expression classifies tumors into clinically relevant groups associated with overall survival. Collagens may be good lineage markers.

Collagen tumor patterns are distinct from normal tissue extracellular matrix and collagen expression patterns.

Collagen clusters are associated with specific cancer genomes.

Clusters are enriched for specific mutation patterns

Clusters are enriched for specific copy number alterations.

Clusters associated with high/low aneuploidy

Machine learning predicts genomic features.

Collagen-defined clusters are associated with specific immunoenvironments.

Collagen-defined clusters are enriched with cancer hallmarks.

These data support an understanding where tumors with high collagen type I environments have lower aneuploidy and ploidy levels compared to tumors with higher expression of tissue-specific minor collagens. The classifier is driven by the expression of minor, non-collagen type I collagens that typically have a specific expression in normal tissue and become dysregulated in many tumors.

This EXAMPLE shows that minor collagens are critical components defining disease progression, the cancer genome and should be included in pre-clinical studies to model the actual human tumor environment and to improve drug development.

Preliminary proof-of-concept data for one such minor collagen, COL7A1, has been shown. These findings demonstrate how the classification of tumors by collagens identified strong links between specific cancer genomes and the tumor extracellular matrix.

### Example 2

### Classifying Tumors by Collagen Expression Reveals Microenvironment Genome Interactions.

The inventors used k-means clustering to classify each of the twenty-six cancer types with 100 cases independently. Silhouette and gap statistic analysis identified the optimal number of clusters for each tumor type. Between three-six well-defined clusters were identified for each cancer type. the inventors named these k-means defined clusters, collagen clusters (ColClusters).

To organize the ColClusters, ColClusters were ordered by stroma fraction with ColCluster 1 having the highest median stroma fraction in each tumor type. The difference in stroma fraction across ColClusters was not significant between ColCluster 1 and ColCluster 2 in 14/26 cancer types examined. Only 8/26 cancer types had similar stroma fraction in ColCluster-2 compared to ColCluster 1. Only 3/26 ColCluster 3's had similar stroma fraction compared to their respective ColCluster 1. ColCluster 1's with high stroma fraction were not always the cluster with the highest expression of fibrillar collagens. ESCA-C4 highly expressed fibrillar collagens but had similar stroma fraction compared to the other ColClusters. Many collagens have ten-fold dynamic range across the ColClusters and cancer types, showing clear definition of the ColClusters. Minor collagens such as COL7A1, COL10A1, COL17A1, and collagen type IX have large dynamic ranges. These collagens have very specific expression in normal tissue, but are dysregulated expressed in many cancer types, though often in only a fraction of tumors in each cancer type. COL17A1 helps discriminate BLCA-C2 and BLCA-C4 and the esophageal carcinoma ColClusters. COL17A1 is high in the gastrointestinal (GI) cancers, colon adenocarcinoma (COAD), rectal adenocarcinomas (READ), and stomach adenocarcinoma, likely because it is expressed in normal gastrointestinal cells. Busslinger et al., Cell Reports (2021).

Bladder Urothelial Carcinoma (BLCA-C1 and BLCA-C2) have similar expression of the fibrillar collagens and stroma fraction. BLCA-C2 is marked by COL17A1 expression and includes many squamous tumors. BLCA-C1 is enriched for Epithelial Mesenchymal Transition and angiogenesis hallmark gene sets. BLCA-C2 was enriched for twenty-seven hallmark gene sets compared to four gene sets in BLCA-C1 with five gene sets with similar QuSAGE scores. Bladder urothelial carcinoma (BLCA-C5) is enriched for FGFR3 mutations and is highest for Notch hallmark gene sets, which is consistent with patients in BLCA-C5 having the longest overall survival, because Notch may be a tumor suppressor pathway. BLCA-C3 and BLCA-C4, distinguished by several minor collagens and relatively lower levels of fibrillar collagen expression. BLCA-C3 was enriched for bile acid metabolism, while BLCA-C4 was enriched for cell cycle regulation and had the shortest overall survival among the bladder urothelial carcinoma ColClusters. Human chromosome 8p loss in all clusters except BLCA-C5. Neuroendocrine tumors were grouped in BLCA-C4 and enriched in BLCA-C5. Papillary and non-papillary forms were biased across ColClusters (p=5⁻¹⁰). High aneuploidy bladder urothelial carcinoma tumors in the ColClusters were not associated with overall survival, while low aneuploidy bladder urothelial carcinoma tumors. Because bladder urothelial carcinoma includes several known histologies, the inventors tested enrichments of the reported histology and assigned mRNA based classifications and found strong enrichment in the ColClusters further linking collagen expression with known classifications and histologies See the decision tree in FIG. 4A.

Breast Invasive Carcinoma (BRCA-C3) was highest for fibrillar collagen expression while BRCA-C1 was enriched for several collagens. BRCA-C2 and BRCA-C5 were marked by COL2A1 expression, with expression of COL4A3/4A4, COL9A1/COL9A3 and COL11A1 discriminating BRCA-C2. BRCA-C4 also had relatively high COL9A1, COL9A3, and COL11A2 expression, but low COL2A1 expression. Breast invasive carcinoma ColClusters were not significantly associated with overall survival, likely because of the long survival times achieved by many patients. KMT2C truncation, PIK3CA missense, TP53 missense, and TP53 truncation variants were significantly biased across breast invasive carcinoma ColClusters. TP53 was localized to BRCA-C2 and BRCA-C4 while PIK3CA was enriched in BRCA-C1 and BRCA-C3. ER⁺ tumors were present in all five breast invasive carcinoma ColClusters, but triple negative tumors dominated BRCA-C4 while ER⁺/HER2+ tumors were prominent in BRCA-C3. These findings show that similar extracellular matrices were somewhat independent of hormone and HER2+ status. Several chromosome arms were enriched in ColClusters including human chromosome 6p gain in BRCA-C2 and losses in human chromosome 12q, 14q, and 15q, in BRCA-C2 and BRCA-C4. Human chromosome 16p gain was enriched in BRCA-C1, BRCA-C3, and BRCA-C5. Pathways to consider for targeting in specific collagen environments include DNA repair, E2F, and Myc in BRCA-C2 and BRCA-C4, consistent with higher proliferation in tumors with more chromosome loss. Epithelial mesenchymal transition was highest in BRCA-C3 marked by high COL10A1 expression. Notch hallmark genet set was highest in BRCA-C5. See the decision tree in FIG. 4B.

Endocervical Adenocarcinoma (CESC) ColClusters were marked by a high fibrillar collagen group, CESC-C1. CESC-C2 is marked by COL4A5/A6, COL7A1, COL16A1, COL17A1 and is enriched for squamous carcinomas. CESC-C3 was marked by collagen type IX and was associated with the longest overall survival, while CESC-C1 and CESC-C2 had similar overall survival curves. CESC-C1 and CESC-C3 were enriched for missense mutations in PIK3CA, which as the only identified biased significantly enriched somatic mutation. Several chromosome arm level copy number alterations were enriched in CESC-C2 compared to the other two CESC ColClusters. These include the infrequent human chromosome 1p, 4q, 19p loss along with the more frequent human chromosome 5p gain and human chromosome 8p loss. Human chromosome 20p and human chromosome 20q gain were enriched in CESC-C1. Human chromosome 18p gain was enriched in CESC-C3. Low frequency amplifications in CCND1, EGFR, the FGF locus, and TERC were enriched in CESC-C2. CESC-C2 was enriched for thirty-one hallmark gene sets including DNA repair. CESC-C1 was enriched for Notch signaling and angiogenesis. Endocervical adenocarcinoma ColClusters had similar immunotype profiles with a mixture of “Wound Healing” and “IFNγ”. CESC-C2 was enriched for several immune cell types including gamma delta T cells, neutrophils, and T helper cells. Effector memory T (Tₑₘ) cells were enriched in CESC-C1. Endocervical adenocarcinoma ColClusters had similar distributions of aneuploidy.

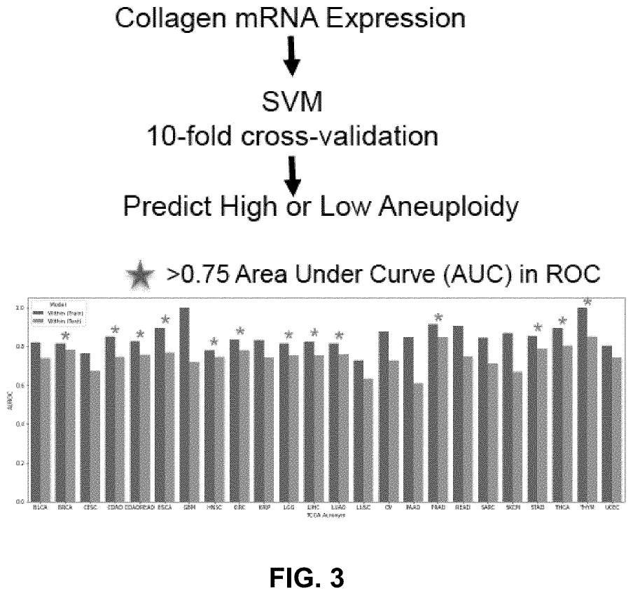

Colon Adenocarcinoma (COAD) ColClusters were associated with overall survival with COAD-C4 including the longest surviving patients. COAD-C1 was marked by high expression of fibrillar collagens. COAD-C2 was marked by COL9A1. High expression of COL2A1 and COL9A3 defined COAD-C3. COL4A5/6 marked COAD-C4. MSIH tumors were enriched in COAD-C1. KRAS mutations were biased towards COAD-C2 and COAD-C3. APC truncations were enriched in COAD-C3. None of the evaluated gene level copy number alterations were biased in the colon adenocarcinoma ColClusters. COAD-C2 was enriched for high aneuploid tumors which also manifested in chromosome arm level copy number alterations enriched in COAD-C2 Hallmark gene sets were highest in COAD-C1 including strong epithelial mesenchymal transition enrichment, Hedgehog signaling, hypoxia, and the inflammatory gene sets. The Wnt signaling hallmark was enriched in COAD-C3. Peroxisome and protein secretion were enriched in COAD-C4. COAD-C2, COAD-C3, and COAD-C4 were enriched for the “Wound Healing” immunotype. COAD-C1 was distributed between “Wound Healing” and “IFNγ”. COAD-C1 was enriched for the majority of immune cell signatures tested. COAD-C3 was enriched for activated CD8 T cells while COAD-C2 and COAD-C3 were not enriched for any immune cell signatures. The Support Vector Machine predicted aneuploidy well with AUC=0.77. See the decision tree in FIG. 3D.

Colorectal Carcinoma (COADREAD). Four COADREAD ColClusters were identified and were significantly associated with overall survival. COADREAD-C1 and COADREAD-C2 were both defined by high fibrillar collagen expression with COADREAD-C2 showing a little bit lower average expression of each fibrillar collagen. COL9A1 expression in COADREAD-C2 and COL9A2 expression in COADREAD-C1 also discriminated these two ColClusters. Relatively high COL2A1 expression defined COADREAD-C3. Relatively high COL4A5/6 expression defined COADREAD-C4. COL9A2, COL9A3, COL11A2, and COL28A1 were also relatively high in COADREAD-C4. Most MSI tumors were in COADREAD-C1. KRAS missense mutations were mildly enriched in COADREAD-C2 and COADREAD-C4. Several low frequency somatic mutations showed mild biases across the ColCluster. No gene level copy number alterations were enriched in specific ColClusters. Several colorectal carcinoma ColClusters were specifically enriched for chromosome arm level copy number alterations. COADREAD-C1 was not as enriched as other ColClusters for copy number alterations. COADREAD-C2 and COADREAD-C3 were different in their collagen expression. They had similar chromosome arm level copy number alterations including human chromosome 8p, 18p, and 18q losses and human chromosome 20q gains. human chromosome 1p and 14q loss, MSI tumors 2p and 2q gain were specific for COADREAD-C3. The Support Vector Machine predicted chromosome arm level copy number alterations COADREAD-C1 was enriched for the majority of hallmark gene sets by QuSAGE, including the inflammation related hallmarks (Allograft Rejection, Inflammatory Response, etc.). COADREAD-C2, even with lower overall levels of fibrillar collagen, was enriched for epithelial mesenchymal transition and hedgehog signaling. COADREAD-C3 was enriched for Wnt Beta Catenin signaling. COADREAD-C4 was enriched for Oxidative Phosphorylation. No ColCluster was enriched for many of the proliferation related hallmarks. COADREAD-C2, COADREAD-C3, and COADREAD-C4 were enriched for the “Wound Healing” immunotype, while COADREAD-C1 included tumors with both “Wound Healing” and “IFNγ”. COADREAD-C1 was also enriched for multiple immune cell signatures by QuSAGE including cytotoxic cells, macrophages, and neutrophils. COADREAD-C2 was enriched for CD56_{dim} cells, showing a more immunocompetent environment. COADREAD-C3 was enriched for Activated CD8 T cells and Effector Memory T cells. COADREAD-C4 was relatively low for immune cell signatures. Aneuploid tumors were enriched in all the ColClusters except COADREAD-C1. The Support Vector Machine performed poorly for colorectal carcinoma with AUC=0.61. Aneuploidy was associated with overall survival in colorectal carcinoma. Stratification by aneuploidy showed that tumors with low aneuploidy in the ColClusters were significantly associated with outcomes, but not tumors with high aneuploidy. MSI tumors were localized to specific ColClusters and the relatively modest differences in collagen expression between COADREAD-C1 and COADREAD-C2 were associated with overall survival, distinct phenotypic states, and immunoenvironments. COADREAD-C3 and COADREAD-C4 were defined by expression of specific collagens. See the decision tree in FIG. 4E.

Esophageal Carcinoma (ESCA) ColClusters were not significantly associated with overall survival. Esophageal carcinoma was distinguished from other cancers in the PanCancer clustering by the expression of COL17A1, a squamous cell marker. ESCA-C1 was defined by modest fibrillar collagen expression, notably COL5A1/2 with COL4A3/4 expression. ESCA-C2 was defined by COL4A5/6 expression. Collagen type IX was highly expressed in ESCA-C1, ESCA-C2, and ESCA-C3. ESCA-C3 was marked by a combination of low fibrillar collagen, high COL4A3/4, and high collagen type IX expression. ESCA-C4 was defined by the high expression of the fibrillar collagens and was enriched for macrophages and regulatory T cells, showing a more immunosuppressive environment. Dendritic cells were also enriched in ESCA-C4, ESCA-C2, and ESCA-C4 were lowest for cytotoxic T cell signatures. Relatively low frequency somatic mutations including KMT2D truncations in ESCA-C2, LRP1B in ESCA-C1, ESCA-C2, and ESCA-C3. PREX2 missense in ESCA-C3 show significant bias in esophageal carcinoma ColClusters. NF1 truncations were enriched in ESCA-C1. ESCA-C2, and ESCA-C4 were enriched for Copy number alterations in several oncogenes and suppressors but were not so different in human chromosome arm level Copy number alterations compared to the other ColClusters. ESCA-C4 was enriched for the most hallmark gene sets. ESCA-C3 was enriched for KRAS signaling. No differences in aneuploidy were observed across the esophageal carcinoma ColClusters. Collagen expression predicts aneuploidy levels. Although aneuploidy was not associated with overall survival across all cases, high aneuploidy tumors in ESCA-C1 had significantly shorter overall survival. See the decision tree in FIG. 4F.

Glioblastoma Multiforme (GBM). The four glioblastoma multiforme ColClusters were not significantly associated with overall survival. Glioblastoma multiforme ColClusters were not as well-defined as ColClusters in other cancer types. GBM-C1 is the high fibrillar collagen expression ColCluster. Glioblastoma multiforme IDH1 mutant tumors were grouped with brain lower grade glioma tumors in the PanCan collagen clustering, showing similar extracellular matrix environments, that were distinct from IDH1 wild-type tumors. GBM-C1 was enriched for multiple immune cell expression signatures including macrophages, mast cells, neutrophils, and T regs. The other glioblastoma multiformes ColClusters were not as infiltrated with immune cells. All glioblastoma multiformes were in the “Lymphocyte Depleted” group. TP53 alterations were enriched in GBM-C2 and GBM-C3. Specific glioblastoma multiforme ColClusters were enriched for several arm level copy number alterations including human chromosome 22q loss, 13q loss, and 14q loss in GBM-C2. Human chromosome 9p loss along with gains in human chromosomes 19p, 19q, 20p, and 20q were enriched in GBM-C4. Glioblastoma multiforme did not show significantly show aneuploidy score biases. Aneuploidy could be predicted from collagen expression with AUC=0.8. This prediction is likely not strong due to the modest number of high aneuploid tumors in the glioblastoma multiforme cohort. The Support Vector Machine predicted chromosome arm level copy number alterations showing a connection between the extracellular matrix and specific genetic alterations. See the decision tree in FIG. 4 G.

Many of the tumor types have high association with survival. Some tumor types do not, such as glioblastoma multiforme.

Head and Neck Squamous Cell Carcinoma (HNSC). Three head and neck squamous cell carcinoma ColClusters were identified and associated with overall survival with HNSC-C3 associated with the longer overall survival. HNSC-C1 is the high fibrillar collagen expression ColCluster. HNSC-C2 has relatively low collagen expression, while HNSC-C3 tumors were enriched for COL4A3/4, collagen type IX, COL19A1, COL21A1, and COL23A1. HNSC-C1 and HNSC-C2 were enriched for P53 missense and truncation variants. Relatively low frequency NSD1 truncations were enriched in HNSC-C2. CDNK2A truncations and FAT1 truncations were enriched in HNSC-C1 and HNSC-C2 and were largely absent from HNSC-C3. HNSC-C3 was enriched for PTEN loss. Copy number gains in EGFR, and losses in CDKN2A and CDKN2B, were enriched in HNSC-C1 and HNSC-C2. HNSC-C1 and copy number HNSC-C2 were enriched for a similar pattern of chromosome arm level Copy number alterations including losses in human chromosome 3p, 4p, 4q, 8p, 9p, 18q, and 21q along with gains in human chromosome 7p and 8q, showing genetic similarity. HNSC-C3 was enriched for gains in human chromosome 8p, 18q, 19p, and 19q and losses in human chromosome 11q, and 16q showing a genetically distinct group of tumors. HNSC-C1 was strongly enriched for epithelial mesenchymal transition, angiogenesis, and myogenesis, while HNSC-C2 and C3 were strongly negatively enriched for these hallmarks. Specific targetable pathways include Hedgehog signaling in HNSC-C1. HNSC-C2 was enriched for hypoxia, glycolysis, mTORC signaling, MYC targets, oxidative phosphorylation, and P53 signaling. HNSC-C3 was negatively enriched for many hallmarks but positively enriched for E2F targets, showing a distinct mechanisms of proliferation compared to the tumors in the other ColClusters. Head and neck squamous cell carcinoma tumors were largely in the IFNγ immunotype, with HNSC-C1 also including some tumors with “Wound Healing”. HNSC-C1 appears wot be more immunosuppressive with enrichment for signatures for eosinophils, macrophages, neutrophils, and regulatory T cells. HNSC-C2 and HNSC-C3 were more immunocompetent environments, in particular HNSC-C3 with enrichment of Activated CD 8 T cells, B Cells, and Cytotoxic cells. HNSC-C3 was enriched for tumors with lower levels of aneuploidy. HNSC-C1 and HNSC-C2 had relatively higher levels of aneuploid tumors compared to HNSC-C3. The Support Vector Machine predicted aneuploidy with moderate success (AUC=0.73). Stratification by aneuploidy revealed that ColClusters for tumors with low aneuploidy were significantly associated with overall survival. ColClusters for tumors with high aneuploidy were not. See the decision tree in FIG. 4H.

Kidney Renal Clear Cell Carcinoma (KIRC). Three kidney renal clear cell carcinoma ColClusters were identified and associated with overall survival with KIRC-C1 associated with the shortest and KIRC-C2 the longest overall survival. KIRC-C1 is the high fibrillar collagen expression group. Truncations in PBRM1 were localized to KIRC-C1 and KIRC-C2. Some KIRC-C3 tumors were in the lymphocyte-depleted immunogroup. KIRC-C1 was enriched for the most hallmark gene sets. KIRC-C3 was enriched for Oxidative Phosphorylation, along with DNA repair, G2M checkpoints, and apical surface hallmark gene sets. Notch and hedgehog signaling gene sets were enriched in KIRC-C2. KIRC-C3 was also lower in several immune cell signatures. Neutrophil expression signatures were enriched in KIRC-C2 while several immune cell signatures were enriched in KIRC-C1. KIRC-C2 has lower aneuploidy. See the decision tree in FIG. 4I.

Kidney renal papillary cell carcinoma (KIRP). Six kidney renal papillary cell carcinoma ColClusters were identified and were strongly associated with overall survival. KIRP-C1 and KIRP-C3 were highest for fibrillar collagen expression. KIRP-C2 was marked by high expression of COL2A1 and COL22A1. Collagen type IV expression was a key determinant of kidney renal papillary cell carcinoma ColClusters. KIRP-C3 highly expressed COL4A5/6 while KIRP-C1 was highest for COL4A1/2 expression. KIRP-C5 was marked by high expression of COL4A5/6. KIRP-C5 had low expression of both fibrillar and type IV collagens, but relatively high expression of collagen type IX. The only significantly biased gene mutation was Met missense variants, enriched in KIRP-C4 and KIRP-C5. Some common gene copy number alterations were observed including CDK6 and EGFR gains enriched in KIRP-C2, KIRP-C4, KIRP-C5, and KIRP-C6. CDKN2A, CDKN2B, and MTAP losses and CCND1 gains had similar patterns enriched in KIRP-C3. KIRP-C3 stands out as distinct when compared to the other kidney renal papillary cell carcinoma ColClusters in arm level Copy number alterations. KIRP-C3 was highest for regulatory T cells. KIRP-C4, the ColCluster with the smallest hazard ratios, was highest for mast cells. Reactive oxygen species hallmark gene set was highest in KIRP-C1 and KIRP-C2 and lowest in KIRP-C4. KIRP-C2 was enriched for oxidative phosphorylation and interferon gamma response gene sets while also relatively high in Adipogenesis and low in Angiogenesis. KIRP-C4 was enriched in cholesterol metabolism and IL2 Stat5 signaling. Kidney renal papillary cell carcinoma ColClusters did not show biases for aneuploidy scores. These data identified KIRP-C3 as a group of tumors with short overall survival, high in collagen expression with distinctive genetics. KIRP-C2, KIRP-C4, KIRP-C5, and KIRP-C6 are tumors with distinctive genetics, immunoenvironments, and pathways with longer overall survival through different mechanisms. See the decision tree in FIG. 4J.

Brain Lower Grade Glioma (LGG). Five brain lower grade glioma ColClusters were identified strongly associated with overall survival. LGG-C1 and LGG-C2 had the highest hazard ratios (HR). Fibrillar collagen expression was highest in LGG-C2. LGG-C1 was marked by a combination of expression of COL6A6, COL8A1, COL19A1, COL21A1, COL23A1, COL24A, and COL25A1. The overall mutation rates was similar across all the ColClusters. TP53 alterations were particularly enriched in LGG-C4, with mild enrichment in LGG-C2 and LGG-C3. IDH1 missense alterations were enriched in LGG-C3, LGG-C4, and LGG-C5 with much lower levels in LGG-C1 and LGG-C2. Low frequency EGFR missense mutations were enriched in LGG-C1 and LGG-C2. ATRX truncations were enriched in LGG-C3 and LGG-C4. Several genes were enriched in specific Brain Lower Grade Glioma ColClusters. Of note was the MTAP and p16/CDKN2A copy number loss and EGFR copy number gains in LGG-C1 and LGG-C2. Lower frequency enrichments of SOX2 gains in LGG-C2. Chromosome arm level Copy number alterations also showed significant enrichments in specific ColClusters. Human chromosome 19q losses were strongly enriched in LGG-C5 and mildly enriched in LGG-C1 and C3. These observations connect molecular alterations to specific collagen compositions in the extracellular matrix. LGG-C1 and LGG-C2, highest in collagen expression, and enriched for many known important molecular alterations had the shorter overall survival compared to the other brain lower grade glioma ColClusters. LGG-C1 and LGG-C2 were each enriched for the largest number, nineteen, of hallmark gene sets, including both epithelial mesenchymal transition and proliferation gene sets such as E2F targets. The other brain lower grade glioma ColClusters showed distinct enrichment patterns. LGG-C1 was enriched for fatty acid metabolism, myogenesis, oxidative phosphorylation. LGG-C3 was enriched for adipogenesis, reactive oxygen species, and xenobiotic metabolism gene sets. LGG-C3 and LGG-C4 had relatively lower levels of aneuploidy. The Support Vector Machine model predicted aneuploidy with high accuracy. See the decision tree in FIG. 4K.

Liver Hepatocellular Carcinoma (LIHC). Three liver hepatocellular carcinoma ColClusters were identified but were not significantly associated with overall survival. LIHC-C1 had the highest expression of fibrillar collagens. LIHC-C2 showed high expression of COL2A1 and COL11A2. LIHC-C3 was defined by generally lower collagen expression and modestly higher expression of COL4A5/6. Gene level copy number alterations were not significantly biased across Liver Hepatocellular Carcinoma ColClusters for the common copy number alterations including MYC and NOTCH2. Chromosome arm level copy number alterations were particularly enriched in LIHC-C2. CTNNB1 alterations were strongly enriched in LIHC-C3. LIHC-C2 was enriched for several chromosome arm level copy gains including human chromosome 19q, 20p, and 20q losses in human chromosome 1p, 4q, 8p, 9p, and 16q. Several specific chromosome arm level copy number alterations were predicted by a Support Vector Machine. Each of the liver hepatocellular carcinoma has distinct immunotypes. LIHC-C1 was high in “Inflammatory”, LIHC-C2 was a mixture of “Inflammatory” and “Lymphocyte Depleted” and the majority of LIHC-C3 tumors were “Lymphocyte Depleted”. Liver hepatocellular carcinoma ColClusters were enriched for 25, 9, and 16 hallmark gene sets, respectively. Wnt beta catenin signaling hallmark gene set was enriched in LIHC-C2. Inflammatory and angiogenesis gene sets were enriched in LIHC-C1. DNA repair and proliferation gene sets were enriched in LIHC-C2. Cholesterol metabolism, oxidative phosphorylation, and reactive oxygen species gene sets were enriched in LIHC-C3. Extracellular matrix defined liver hepatocellular carcinoma groups have distinctive features to target. Liver hepatocellular carcinoma ColClusters did not show biases for aneuploidy scores.

Lung Adenocarcinoma (LUAD). The four lung adenocarcinoma ColClusters were significantly associated with overall survival with LUAD-C1 having the shortest overall survival. Fibrillar collagen expression was highest in LUAD-C1. LUAD-C2 was marked by high COL4A3/4/5/6 and COL6A6 expression. LUAD-C3 was marked by COL25A1 expression and LUAD-C4 was marked by COL2A1 and COL11A2 expression. P53 missense and truncation variants were enriched in LUAD-C1 and LUAD-C4 with the fewest fraction of tumors with P53 alterations in LUAD-C3. LUAD-C3 was enriched for KRAS missense variants, although many tumors with KRAS missense mutations were in each lung adenocarcinoma ColCluster. Lower frequency alterations including LRP1B missense mutations were enriched in LUAD-C1 and C4. Copy number gains in CDK4, EGFR, SOX2, TERC, and TERT were enriched in LUAD-C4. Gains in Myc were biased to LUAD-C3, which also was highest for Myc targets hallmark gene set. LUAD-C1 was enriched for the most hallmarks. LUAD-C2 was enriched for several inflammation gene sets including interferon alpha response and interferon gamma response and the P53 pathway. LUAD-C4 was enriched for the E2F targets, G2M checkpoints and DNA repair gene sets. LUAD-C3 was enriched for xenobiotic metabolism, unfolded protein response, oxidative phosphorylation and fatty acid metabolism. Lung adenocarcinoma tumors have diverse immunoenvironments that were biased across the ColClusters. The lung adenocarcinoma ColClusters showed a range of enrichments for immunotypes. LUAD-C1 and LUAD-C4 were enriched for “Wound Healing” and IFNγ while LUAD-C2 and LUAD-C3 were enriched for “Inflammatory” with some tumors enriched for “IFNγ”. Central and effector memory T cells were enriched in LUAD-C2 and LUAD-C3. Regulatory T cells were enriched in LUAD-C1. LUAD-C2 was enriched for lower aneuploidy while LUAD-C4 was enriched for higher aneuploidy compared to LUAD-C1. The Support Vector Machine predicted aneuploidy scores based on collagen expression with high accuracy. See the decision tree in FIG. 4M.

Lung Squamous Cell Carcinoma (LUSC). The six lung squamous cell carcinoma ColClusters were not associated with overall survival. LUSC-C1, LUSC-C2, and LUSC-C3 were all high in fibrillar collagen expression and discriminated by expression of COL4A3/4 (LUSC-C2), collagen type IX (LUSC-C2 and LUSC-C3), COL19A1 (LUSC-C2) and COL21A1, COL22A1, and COL23A1. LUSC-C4 was marked by low fibrillar collagen expression, high COL4A5/6, and COL21A1. LUSC-C5 was marked by low fibrillar collagen expression, high expression of COL4A5/6, COL17A1, COl27A1, and COL28A1. Lung squamous cell carcinoma ColClusters have biased distributions of low frequency somatic mutations in PTEN, PTPRB, PTPRT, and RB1. Genes with high frequency were enriched in all the ColClusters except LUSC-C4. LUSC-C4 was enriched for losses in human chromosome 22q and 19p. LUSC-C3 was enriched for losses in human chromosome 18q. LUSC-C2 was enriched for losses in human chromosome 14q. These copy number alterations define the genetic-extracellular matrix relationships for lung squamous cell carcinoma. No biases were observed for aneuploidy scores in lung squamous cell carcinoma. LUSC-C1 and C3 were each enriched for many hallmarks. LUSC-C2, also relatively high in fibrillar collagen expression, was enriched for only the Allograft Rejection gene set. Along with LUSC-C4, LUSC-C2 was enriched for B cells, Cytotoxic cells. LUSC-C4 was also enriched for Macrophages. These observations show distinct immunoenvironments in each ColCluster. Lung squamous cell carcinoma ColClusters showed no biases for aneuploidy scores. Collagen expression could not predict aneuploidy robustly in lung squamous cell carcinoma. See the decision tree in

Ovarian Serous Cystadenocarcinoma (OV). Three ovarian serous cystadenocarcinoma ColClusters were identified that were not associated with overall survival lung squamous cell carcinoma ColClusters show no biases for aneuploidy scores, nor could collagen expression predict aneuploidy robustly in lung squamous cell carcinoma. OV-C1 was the high fibrillar expression ColCluster. OV-C3 was marked by high COL2A1, COL4A5/6, and COL9A3 expression. OV-C2 has relatively low collagen expression lung squamous cell carcinoma ColClusters show no biases for aneuploidy scores, nor could collagen expression predict aneuploidy robustly in lung squamous cell carcinoma. No somatic variants evaluated showed significant enrichments in an ovarian serous cystadenocarcinoma ColCluster. Lung squamous cell carcinoma ColClusters show no biases for aneuploidy scores, nor could collagen expression predict aneuploidy robustly in lung squamous cell carcinoma. Human chromosome 2p gain was enriched in OV-C3. Human chromosome 12q gain and 9p loss were enriched in OV-C2 and OV-C3. Human chromosome 8p loss was enriched in both OV-C1 and OV-C3. Lung squamous cell carcinoma ColClusters show no biases for aneuploidy scores, nor could collagen expression predict aneuploidy robustly in lung squamous cell carcinoma. OV-C1 was enriched for thirty-eight of the fifty hallmark gene sets. OV-C3 was the most enriched for Epithelial Mesenchymal Transition, Notch, and Wnt Beta Catenin signaling, showing connections between these signaling pathways and the distinct tumor extracellular matrices. Ovarian serous cystadenocarcinoma ColClusters showed no biases for aneuploidy scores, nor could collagen expression predict aneuploidy robustly in lung squamous cell carcinoma. Even for a heterogeneous copy number driven cancer type such as ovarian serous cystadenocarcinoma, distinct connections between the genetics and tumor extracellular matrix can be identified. See the decision tree in FIG. 40.

Pancreatic adenocarcinoma (PAAD). The four identified pancreatic adenocarcinoma ColClusters were significantly associated with overall survival with PAAD-C4 distinct from the other three and PAAD-C1 and PAAD-C2 were both marked by high fibrillar collagen expression and distinguished by differences in COL4A3/4 expression along with differences in COL10A1 and some fibrillar collagens including COL11A1 higher in PAAD-C2 while COL141 and COL15A1 had higher expression in PAAD-C1. PAAD-C3 was marked by high expression of COL9A2, COL9A3, and COL11A2. PAAD-C4 was marked by high expression of COL2A1, COL4A6, and COL25A1. Because PAAD-C1 includes the high stroma fraction and lower tumor cell fraction, these tumors were underrepresented for KRAS and TP53 variants. TP53 and KRAS variants were notably absent in the long surviving PAAD-C4 group. PAAD-C1 was notably not enriched for chromosome arm level Copy number alterations. PAAD-C2 and PAAD-C3 had similar patterns of chromosome level Copy number alterations, mostly chromosome arm copy losses. PAAD-C4 had several chromosome level copy number gains. Many of the chromosome arm level Copy number alterations were predicted from collagen expression by Support Vector Machine. PAAD-C2 was enriched for the most hallmark gene sets. Even though both PAAD-C2 and C2 highly expressed fibrillar collagens, only PAAD-C1 was enriched for the TGFβ gene set. PAAD-C3 was enriched for cholesterol metabolism and oxidative phosphorylation. Aneuploidy scores are borderline significantly biased across PAAD ColClusters, but the Support Vector Machine performed poorly to predict aneuploidy, perhaps because of the only modest separation of the k-means defined clusters. Pancreatic cancer is heterogeneous, but specific links exist between collagen expression and combinations of chromosome level arm copy number alterations. See the decision tree in FIG. 4P.

Pheochromocytoma and Paraganglioma (PCPG). Four pancreatic adenocarcinoma ColClusters were identified and were not associated with overall survival. PCPG-C1 and PCPG-C2 were enriched for fibrillar collagen expression with PCPG-C1 marked by the neuronal collagen, COL20A1. PCPG-C4 was marked by relatively low fibrillar collagen expression and high COL20A1 expression. PCPG-C3 was marked by a combination of low fibrillar collagen expression and high COL4A5/6 expression. Low frequency NF1 truncations were enriched in PCPG-C4. Low frequency HRAS missense variants were enriched in PCPG-C3 and PCPG-C4. None of the evaluated genes showed copy number alteration enrichments. A few chromosome arm level Copy number alterations, mostly copy number losses, were predicted from collagen expression by the Support Vector Machine, showing connections between the genetics and the tumor extracellular matrix. No one pancreatic adenocarcinoma ColCluster was enriched for the large majority of hallmark gene sets. A few hallmark gene sets showed high enrichment in a pancreatic adenocarcinoma ColCluster. Epithelial mesenchymal transition and cholesterol metabolism hallmark gene sets were enriched in PCPG-C2. glycolysis, hypoxia, and mTORC signaling gene sets were enriched in PCPG-C1. Pancreatic Beta Cells gene set was enriched in PCPG-C3. These findings define distinct phenotypic states for tumors in pancreatic adenocarcinoma ColClusters. Pancreatic adenocarcinoma ColClusters did not have significant biases of aneuploidy scores or sufficient numbers to test a Support Vector Machine.

Prostate adenocarcinoma (PRAD). Three prostate adenocarcinoma ColClusters were identified. Prostate Adenocarcinoma patients in the cohort live a long time and no association with overall survival was observed and PRAD-C2 is the high fibrillar expression ColCluster. PRAD-C1 is marked by expression of COL4A5/6, COL7A1, and COL9A1. PRAD-C3 is marked by expression of COL2A1 and COL9A2/3. No variants in the genes evaluated were significantly biased across the ColClusters. Some low frequency copy number alterations in a few genes were significantly enriched in specific prostate adenocarcinoma ColClusters including MYC and RAD21 gains in PRAD-C2, PTEN losses in PRAD-C3 and AGO2 gains in PRAD-C2 and PRAD-C3. Each Prostate Adenocarcinoma ColCluster had distinct enrichment for hallmarks. PRAD-C1 was enriched for androgen response, interferon alpha, and interferon gamma. PRAD-C2 was enriched for angiogenesis, E2F Targets, and fatty acid metabolism. PRAD-C3 was enriched for DNA repair, G2M checkpoints, PI3K AKT mTOR signaling, protein secretion, and unfolded protein response. Prostate Adenocarcinoma ColClusters had distinct immunoenvironments. The three prostate adenocarcinoma ColClusters have similar immunotypes with PRAD-C2 and PRAD-C3 including some tumors with the “Wound Healing” immunotype not observed in PRAD-C1. Both PRAD-C1 and PRAD-C2 were enriched for neutrophils while PRAD-C3 had relatively low levels of neutrophils. PRAD-C1 was enriched for B cells while no significant biases were observed for cytoxic T cells. PRAD-C2 and PRAD-C3 had higher aneuploidy scores than PRAD-C1. The Support Vector Machine predicted aneuploidy based on collagen expression with high accuracy (AUC=0.86). Many chromosome arm level copy number alterations were enriched in specific prostate adenocarcinoma ColClusters including human chromosome 8p loss in PRAD-C3, human chromosome 8q gain in PRAD-C2, and human chromosome 16q loss in PRAD-C2 and PRAD-C3. Stratification by aneuploidy scores did not reveal significant association with overall survival. See the decision tree in FIG. 4R.

Rectum Adenocarcinoma (READ). Three rectum adenocarcinoma ColClusters were identified and were not associated with overall survival. READ-C1 is the high fibrillar collagen expression group. READ-C2 has lower collagen expression and is marked by COL9A1 expression. READ-C3 is marked by COL4A5/6 and COL9A2 expression. APC truncations populated all the READ ColClusters while READ-C3 was most enriched for KRAS missense variants. Relatively few READ-C1 tumors had KRAS mutations. No gene level copy number alterations were enriched in a READ ColCluster. READ-C2 was most enriched for a few chromosome arm level copy number alterations with enrichment for losses in human chromosome 14q and gains in human chromosome 13q, 16q, 16p, 20p and 20q. READ-C1 and READ-C2 had similar enrichments that differed from READ-C3 including losses in human chromosome 1p, 4q and 8p and gains in 7p and 8q. READ-C1 was enriched for the most hallmark gene sets including angiogenesis, epithelial mesenchymal transition, and the inflammatory hallmarks. No hallmarks were enriched in READ-C2. READ-C3 was enriched for rectum adenocarcinoma. Rectum adenocarcinoma ColClusters were all enriched for the “Wound Healing” immunotype while READ-C1 also included tumors with the “IFNγ” immunotype. READ-C1 was enriched for several immune cells consistent with an immunosuppressive environment including macrophages and regulatory T cells. READ-C2 and READ-C3 were not enriched for immune cell signatures. Aneuploidy scores were not biases across the rectum adenocarcinoma ColClusters. The human chromosome predicted high aneuploidy at AUC=0.74 in rectum adenocarcinoma. Stratification by aneuploidy did not reveal associations with overall survival in rectum adenocarcinoma ColClusters. See the decision tree in FIG. 4S.

Sarcoma (SARC). The four sarcoma ColClusters was borderline associated with overall survival with SARC-C4 with lower overall survival. Both SARC-C1 and SARC-C2 had relatively high expression of several fibrillar collagens. SARC-C2 had higher expression of COL7A1, COL8A1, COL10A1 and COL11A1. SARC-C3 was defined by relatively high expression of both COL4A1/2 and COL4A5/6. SARC-C4 was defined by high expression of COL2A1, all three collagen type IX genes (COL9A1, COL9A2, COL9A3), COL11A2, COL20A1, 00123A1, and COL25A1. RB1 truncations and TP53 missense variants were specifically enriched in SARC-C3. No other variants enrichments for the genes evaluated was observed. Many gene level Copy number alterations were significantly biased across the SARC ColClusters. Similar to RB1 truncations, RB1 losses were also enriched in SARC-C3. MYC gains were enriched in SARC-C4. CCNE1 gains were enriched in SARC-C1. Chromosome arm level copy number alterations specific enrichments were prevalent across the SARC ColClusters. Most notably, human chromosome 18q loss and human chromosome 1p gain were enriched in SARC-C2. Human chromosome 10q loss defined SARC-C3. SARC-C1 was defined by copy number gains in several chromosome arms including 17p, 18p, 19p and 19q. Sarcoma ColCluster phenotypes were strongly associated with distinct phenotypes as indicated by QuSAGE enrichment of hallmark gene sets. SARC-C2 was enriched for the most hallmark gene sets with SARC-C1 also enriched for many hallmark gene sets. SARC-C4 was enriched for Notch signaling, unfolded protein response, and Wnt Beta Catenin signaling hallmark gene sets. SARC tumors include a diverse array of immunotypes with SARC-C4 enriched for “Wound Healing” and no “IFNγ”. SARC-C1 and SARC-C2 have some tumors with “TGFβ” and otherwise SARC-C1, SARC-C2, and SARC-C3 include a mixture of four immunotypes. SARC-C3 was enriched for B cells. SARC-C1 was enriched for several immune cells including dendritic. Neutrophils and Tregs were enriched in both SARC-C1 and C2. SARC-C3, and SARC-C4 had relatively low expression of several immune cells including Neutrophils and Tregs. SARC-C4 was enriched for T helper cells. SARC-C3 had lower aneuploidy scores compared to the other three SARC ColClusters. The Support Vector Machine moderately predicted sarcoma high aneuploid tumors (AUC=0.73). Many chromosome arm level Copy number alterations, especially losses, were predicted by Support Vector Machine. Because sarcoma represents a diverse group of tumors from multiple tissue site locations with distinct histologies strongly enriched into specific sarcoma ColClusters. See the decision tree in FIG. 4T.

Skin Cutaneous Melanoma (SKCM). The four identified skin cutaneous melanoma ColClusters were not associated with overall survival ColClusters did not have significant biases of aneuploidy scores or sufficient numbers to test a Support Vector Machine and ColClusters did not have significant biases of aneuploidy scores or sufficient numbers to test a Support Vector Machine. the inventors only evaluated the primary Skin Cutaneous Melanoma tumors because the tumor microenvironment and extracellular matrix would be expected to differ greatly in metastases and therefore observations are limited in skin cutaneous melanoma because of the small cohort. Skin cutaneous melanoma ColClusters were weakly defined. SKCM-C1 and SKCM-C2 had relatively high fibrillar collagen expression. SKCM-C3 was defined by COL2A1. SKCM-C4 was marked by generally low heterogeneous collagen expression. No somatic mutations in the genes tested, gene level nor chromosome arm levels copy number alterations tested were significantly biased across the skin cutaneous melanoma ColClusters. Skin cutaneous melanoma ColClusters did not have significant biases of aneuploidy scores or sufficient numbers to test a Support Vector Machine. The Support Vector Machine did not predict aneuploid tumors. The Support Vector Machine predicted specific chromosome arm level copy number alterations for several arm level gains and losses. No hallmark gene sets were significantly enriched compared to the other ColClusters for hallmark gene sets. SKCM-C2 was notable for low enrichment of mTORC1 signaling and oxidative phosphorylation hallmark gene sets. Skin Cutaneous Melanoma ColClusters included tumors from four immunotypes. Likely because of low numbers, QuSAGE did not identify enriched immune cell signatures in the skin cutaneous melanoma ColClusters. Aneuploidy scores were not biases across the skin cutaneous melanoma ColClusters. The Support Vector Machine predicted high aneuploidy at only AUC=0.65 in skin cutaneous melanoma. Stratification by aneuploidy scores did not reveal significant association with overall survival. See the decision tree in FIG. 4U.

Stomach Adenocarcinoma (STAD) ColClusters were associated with overall survival. STAD-C3, enriched for aneuploid tumors and along with the high collagen STAD-C1, had the shortest overall survival. STAD-C1 and STAD-C2 both highly expressed fibrillar collagens with STAD-C1 higher for COL3A1 and collagen type IV and STAD-C2 higher for COL5A1/2 and COL11A1. STAD-C3, the high aneuploidy ColCluster was marked by high COL2A1 expression. High COL9A3 and COL11A2 expression mark STAD-C5. STAD-C4, with high expression of COL11A2 and COL9A3, was enriched for APC truncations and had the highest levels of Wnt signaling by QuSAGE. ARID1A mutations were enriched in STAD-C1, STAD-C2, and STAD-C4, but not in the high aneuploidy STAD-C3 and STAD-C5 groups. MSI cases were largely grouped in STAD-C2. P53 missense variants were enriched in STAD-C3. STAD-C3 and STAD-C5 were enriched for many gene and arm level copy number alterations. No other significantly biased copy number alterations were enriched in the other ColClusters. Tumors with three immunotypes were significantly populated in STAD-C1. STAD-C2, STAD-C4, and STAD-C5 has similar distributions between “Wound Healing” and “IFNγ”, while the majority of STAD-C3 tumors were in “Wound Healing”. STAD-C1 had high expression levels of many of the immune cell signatures. STAD-C2 was highest for activated Dendritic Cells (aDC). STAD-C3 had the lowest levels of B cells and cytotoxic cells. STAD-C4 was highest for NK cells. STAD-C5 was enriched for Wnt Beta Catenin signaling. Angiogenesis was highest in STAD-C1 and STAD-C2, which were the two high fibrillar and collagen type IV Stomach adenocarcinoma ColClusters. STAD-C3 and STAD-C5 were enriched for aneuploid tumors. The Support Vector Machine predicted aneuploidy in stomach adenocarcinoma tumors with high accuracy. Genetically similar stomach adenocarcinoma tumors were distinguished by their collagen expression patterns. See the decision tree in FIG. 4V.

Testicular Germ Cell Tumors (TGCT). Four testicular germ cell tumors ColClusters were identified that were not associated with overall survival as the large majority of patients all had long overall survival and. TGCT-C4 was high in fibrillar collagen, low in stroma fraction, and enriched for AGO2, MYC, and RAD21 copy number gains. KRAS amplifications were high in each testicular germ cell tumors ColCluster except TGCT-C1. TCGT-C1 was enriched for KIT and KRAS missense mutations. TCGT-C3 had the second expression levels of fibrillar collagens. TGCT-C1 and TGCT-C2 were marked by expression of COL6A6, COL17A1, COL22A1, COL23A1, and the neuronal specific collagen, COL20A1. The collagen type IV genes were key discriminators. COL4A5/6 was high in TGCT-C4 and TGCT-C1 but not TGCT-C2 and TGCT-C3. Several chromosome arm levels copy number alterations were enriched in specific testicular germ cell tumors ColClusters. Human chromosome 1q, 12q, and 22q gains were enriched in TGCT-C2, while 22q losses were enriched in TGCT-C4. Several chromosome arm level copy number alterations had biased distribution across the Testicular Germ Cell Tumor ColClusters. TGCT-C3 and TGCT-C4 were enriched for nineteen and twenty-five hallmark gene sets, respectively. TGCT-C1 was enriched for allograft rejection, interferon alpha, interferon gamma, and KRAS signaling up gene sets. TGCT-C4 was enriched for the “Wound Healing” immunotype, while the other three TGCT ColClusters were enriched for “IFNγ” immunotype. No biases in aneuploid tumors were observed across the testicular germ cell tumors ColClusters. TGCT-C1 and TCGT-C2 were enriched for several immune cells while TGCT-C4 was not, except for mast, regulatory T, and iDC cells. These observations show that genetically distinct tumors were associated with distinctive collagen defined extracellular matrices in testicular germ cell tumors. See the decision tree in FIG. 4W.

Thyroid Carcinoma (THCA). Four thyroid carcinoma ColClusters were identified that were modestly associated with overall survival. Thyroid carcinoma ColClusters were defined by stark differences in collagen expression. THCA-C1 is defined by fibrillar collagen expression including collagen types I and V along with COL10A1, COL11A1, COL12A1, COL22A1, and COL24A1. THCA-C2 had relatively low collagen expression while THCA-C3 was marked by COL4A1/2, COL4A5/6, and COL9A3. THCVA-C4 was defined by COL4A5/6, COL6A6, and COL9A1. BRAF missense mutations were enriched in THCA-C1, THCA-C2 and THCA-C3. Only one THVCA tumor in THCA-C3 had a BRAF mutation. NRAS missense variants were enriched in THCA-C4 with a few tumors with NRAS missense in THCA-C3. Only one thyroid carcinoma tumor in THCA-C1 or THCA-C2 had a NRAS missense mutation. Low frequency EGFR amplifications were enriched in THCA-C3. Chromosome arm level copy number alterations were not frequent in thyroid carcinoma. Human chromosome 22q loss was enriched in THCA-C4. Human chromosome 12p, 12q, 5p, 5q, 7p, and 7q gains were enriched in THCA-C3. Human chromosome 1q gain was enriched in THCA-C1. THCA-C1 was enriched for the most hallmark gene sets. THCA-C1 and C2 had similar hallmark enrichment patterns. THCA-C3 and THCA-C4 were enriched for similar hallmarks. THCA-C1 was strongly enriched for angiogenesis. Both THCA-C1 and THCA-C2 were enriched for several inflammation-related hallmark gene sets along with epithelial mesenchymal transition and cholesterol metabolism. Fatty acid metabolism, oxidative phosphorylation, and mTORC signaling were enriched in THCA-C3 and THCA C4. Aneuploidy scores were not biased across the thyroid carcinoma ColClusters. The Support Vector Machine predicted aneuploidy with AUC=0.83. The Support Vector Machine predicted the copy number alteration for two chromosome arm level gains and three losses. See the decision tree in FIG. 4X.

Thymoma (THYM). Three thymoma ColClusters were identified that were not associated with overall survival. THYM-C2 was the high fibrillar collagen expression ColCluster. THYM-C3 also included high expression of some fibrillar collagens along with COL8A2 and COL28A1. THYM-C1 had relatively low expression of collagens. THYM-C2 had higher overall mutation rates, but no gene with mutations was enriched in a ColCluster. Only low frequency gene level copy number alterations were observed and localized to THYM-C2. Interestingly, several chromosome arm level copy number alterations were localized to THYM-C2. The modest genetic enrichments were complemented by strong phenotypic enrichments in the Thymoma ColClusters. THYM-C2 was enriched for inflammatory gene sets including inflammatory response and IL6 JAK STAT3 signaling hallmark gene sets. Wnt Beta Catenin signaling and TGFβ were enriched in THYM-C3. Aneuploidy scores were lower in THYM-C3. The Support Vector Machine predicted aneuploidy scores with high accuracy. There were fewer than ten high aneuploid cases in the Thymoma cohort. Several chromosome arm level copy number gains and losses were predicted by collagen expression including human chromosome 1q gain and 11p loss. No immunotypes were reported for thyoma. Activated CD8 T cell expression signature was enriched in THYM-C1. B cell. Neutrophil expression signatures were enriched in THYM-C2. THYM-C3 was enriched for more immunosuppressive cells including macrophages and T regulatory cells. See the decision tree in FIG. 4Y.

Uterine Corpus Endometrial Carcinoma (UCEC). The four Uterine Corpus Endometrial Carcinoma ColClusters were associated with overall survival. UCEC-C1 was the high fibrillar collagen expression ColCluster. UCEC-C2 is the low collagen expression ColCluster. UCEC-C3 was defined by COL2A1 and COL21A1 expression. UCEC-C4 was defined by COL8A2, collagen type IX, COL19A1, COL22A1, COL23A1, and COL25A1 expression. UCEC-C1, UCEC-C2, and UCEC-C3 have relatively high mutation rates compared to UCEC-C4. PTEN truncations were enriched in UCEC-C1, UCEC-C2, and UCEC-C3. P53 missense mutations were enriched in UCEC-C4. ARID1A truncations were enriched in UCEC-C1 and UCEC-C3. PIK3CA missense mutations were enriched in UCEC-C2. Many gene and chromosome level copy number alterations were enriched in UCEC-C4. This ColCluster has high aneuploidy and polyploidy compared to the other UCEC ColClusters. The Support Vector Machine predicted high aneuploid tumors with AUC=0.74. The Support Vector Machine predicted many chromosome level copy number alterations with high accuracy. UCEC-C4 had a distinct distribution of immunotypes with more “IFNγ.” The other Uterine corpus endometrial carcinoma ColClusters had more “Wound Healing” immunotypes. UCEC-C3 was enriched for bile acid metabolism and protein secretion. The high aneuploid, shorter survival UCEC-C4 was enriched for DNA repair, E2F targets, G2M checkpoints, hedgehog signaling, and Notch signaling. See the decision tree in FIG. 4Z.

TABLE 2
RSEM scores for each collagen gene in
each tumor type in each ColCluster

| | | 0.05 | 0.95 | |
|---|---|---|---|---|
| Characteristic | HR | Cl | Cl | p |
| BLCA-C1 | 1.00 | | | |
| BLCA-C2 | 0.96 | 0.66 | 1.38 | 8.1E−01 |
| BLCA-C3 | 0.78 | 0.50 | 1.21 | 2.7E−01 |
| BLCA-C4 | 1.62 | 0.90 | 2.88 | 1.1E−01 |
| BLCA-C5 | 0.55 | 0.33 | 0.94 | 3.0E−02 |
| BLCA.Stromal.Fraction | 1.93 | 0.98 | 3.79 | 5.7E−02 |
| BLCA.pStageII | 2104467.97 | 0.00 | inf | 9.9E−01 |
| BLCA.pStageIII | 3255994.00 | 0.00 | inf | 9.9E−01 |
| BLCA.pStageIV | 5861811.23 | 0.00 | inf | 9.9E−01 |
| BRCA-C1 | 1.00 | | | |
| BRCA-C2 | 0.64 | 0.25 | 1.63 | 3.5E−01 |
| BRCA-C3 | 1.11 | 0.74 | 1.68 | 6.1E−01 |
| BRCA-C4 | 1.04 | 0.59 | 1.84 | 8.9E−01 |
| BRCA-C5 | 1.07 | 0.65 | 1.75 | 7.9E−01 |
| BRCA.Stromal.Fraction | 0.83 | 0.34 | 2.05 | 6.9E−01 |
| BRCA.pStageII | 1.60 | 0.91 | 2.80 | 1.0E−01 |
| BRCA.pStageIII | 3.11 | 1.73 | 5.60 | 1.5E−04 |
| BRCA.pStageIV | 9.39 | 4.54 | 19.40 | 1.5E−09 |
| CESC-C1 | 1.00 | | | |
| CESC-C2 | 0.99 | 0.56 | 1.72 | 9.6E−01 |
| CESC-C3 | 0.47 | 0.25 | 0.87 | 1.6E−02 |
| CESC.Stromal.Fraction | 0.34 | 0.07 | 1.50 | 1.5E−01 |
| CESC.cStageII | 0.91 | 0.46 | 1.81 | 8.0E−01 |
| CESC.cStageIII | 1.32 | 0.63 | 2.76 | 4.6E−01 |
| CESC.cStageIV | 4.83 | 2.58 | 9.05 | 8.5E−07 |
| COAD-C1 | 1.00 | | | |
| COAD-C2 | 0.85 | 0.48 | 1.52 | 5.8E−01 |
| COAD-C3 | 0.79 | 0.34 | 1.86 | 5.9E−01 |
| COAD-C4 | 0.35 | 0.17 | 0.75 | 6.9E−03 |
| COAD.Stromal.Fraction | 3.09 | 0.78 | 12.17 | 1.1E−01 |
| COAD.pStageII | 1.78 | 0.52 | 6.12 | 3.6E−01 |
| COAD.pStageIII | 3.51 | 1.04 | 11.81 | 4.3E−02 |
| COAD.pStageIV | 9.47 | 2.78 | 32.20 | 3.2E−04 |
| COADREAD-C1 | 1.00 | | | |
| COADREAD-C2 | 0.60 | 0.35 | 1.05 | 7.4E−02 |
| COADREAD-C3 | 0.89 | 0.47 | 1.66 | 7.1E−01 |
| COADREAD-C4 | 0.31 | 0.16 | 0.62 | 9.2E−04 |
| COADREAD.Stromal.Fraction | 2.33 | 0.64 | 8.50 | 2.0E−01 |
| COADREAD.pStageII | 1.04 | 0.38 | 2.84 | 9.3E−01 |
| COADREAD.pStageIII | 2.54 | 0.98 | 6.59 | 5.5E−02 |
| COADREAD.pStageIV | 5.52 | 2.07 | 14.73 | 6.5E−04 |
| ESCA-C1 | 1.00 | | | |
| ESCA-C2 | 0.85 | 0.38 | 1.90 | 6.9E−01 |
| ESCA-C3 | 0.73 | 0.39 | 1.38 | 3.4E−01 |
| ESCA-C4 | 0.92 | 0.48 | 1.78 | 8.1E−01 |
| ESCA.Stromal.Fraction | 0.75 | 0.17 | 3.26 | 7.1E−01 |
| ESCA.pStageII | 1.81 | 0.68 | 4.79 | 2.3E−01 |
| ESCA.pStageIII | 4.26 | 1.57 | 11.55 | 4.4E−03 |
| ESCA.pStageIV | 9.58 | 2.89 | 31.71 | 2.2E−04 |
| GBM-C1 | 1.00 | | | |
| GBM-C2 | 0.97 | 0.51 | 1.85 | 9.4E−01 |
| GBM-C3 | 0.83 | 0.51 | 1.37 | 4.7E−01 |
| GBM-C4 | 0.77 | 0.47 | 1.26 | 3.0E−01 |
| GBM.Stromal.Fraction | 1.75 | 0.53 | 5.78 | 3.6E−01 |
| HNSC-C1 | 1.00 | | | |
| HNSC-C2 | 1.07 | 0.81 | 1.42 | 6.2E−01 |
| HNSC-C3 | 0.39 | 0.20 | 0.74 | 4.2E−03 |
| HNSC.Stromal.Fraction | 0.89 | 0.39 | 2.03 | 7.8E−01 |
| HNSC.cStageII | 1.01 | 0.45 | 2.27 | 9.7E−01 |
| HNSC.cStageIII | 1.19 | 0.53 | 2.64 | 6.7E−01 |

### Analysis of Results

Collagen mRNA expression in bulk tumor samples is a result of a complicated contribution from multiple cell types including fibroblasts, macrophages, and tumor cells. Naba et al., Journal of Proteome Research (2017). The inventors evaluated the relationship between the stroma fraction, the ColClusters, and collagen expression to test if collagen composition was correlated with stroma fraction. The relationship between collagens and stroma fraction varies in each tumor setting. As collagen type I is the dominant collagen secreted by fibroblasts and stroma cells, COL1A1 is positively correlated with stroma in all but three of the cancer types. Stroma and collagen expression were also strongly positively correlated for many of the other fibrillar collagens including collagen types III, V, XI, and XIV, regulators of collagen type I fiber width and structure. See Ricard-Blum, Cold Spring Harbor Perspectives in Biology (2011).

Even in ColClusters with similar stroma fraction, significant collagen expression differences showed that collagen composition and stroma fraction are distinct characteristics. Many of the non-fibrillar collagens including collagen types VII, VIII, IX, COL4A5, COL4A6, and others, were only modestly correlated with stroma fraction.

The brain specific collagen, COL20A1, was only significantly expressed in neuronal lineage tumors (glioblastoma multiforme, brain lower grade glioma, pancreatic adenocarcinoma, and TGCT).

COL25A1 is dysregulated and expressed in kidney renal clear cell carcinoma, lung adenocarcinoma, sarcoma, thyroid carcinoma, and UCEC cancer types.

Other high dynamic range collagens including collagen type IX (COL9) and COL4A5/6 marked specific ColClusters. COL10A1 and COL4A5/6 helped define SARC-C4 and TGCT-C1.

Six genes express collagen type IV which is the major component of the basement membrane. Each pair of collagen type IV's (COL4A1/A2, COL4A3/A4, and COL4A5/A6) are co-regulated from shared divergent promoters. Collagen type IV shows a large dynamic range of expression both across and within cancer types. These pairs of collagen type IV genes have distinctive expression patterns in each cancer type and in the ColClusters. Twenty-six of the 104 ColClusters were defined by high expression of one of the COL4 pairs including in all cancer types except Prostate Adenocarcinoma. COL4A1/A2 and COL4A3/A4 have distinct phenotypes in mice. See Cosgrove et al., Genes & Development (1996). These observations show differential functions in these tumors.

Overall Survival. 13/26 of the cancer type ColClusters were significantly associated with overall survival with p-values. Kaplan-Meier curves showed the separation of high and low risk patients for ColClusters. Univariate Cox proportional hazards were derived from hazard ratios in each cluster. Notably, 15/26 of ColCluster-1's, with the highest stroma fraction in each cancer type, were associated with high risk hazard ratios (HR). Among these, 7/13 cancer types had ColClusters with worse or similar hazard ratio as ColCluster-1 had significantly lower stromal fraction, showing that the collagen composition, independent of stroma, was important for patient outcomes.

Multivariate cox proportional hazards analysis showed that ColClusters were independent of stroma fraction and staging. All together, these observations show that the specific composition of collagen-defined tumor extracellular matrix was associated with overall survival in multiple cancer types, independent from the total stroma fraction and staging.

Collagen clustering identifies tissue of origin. Collagens have been used as biomarkers for specific cell types and cell states. These findings show that collagens distinguish cancer types by their tissues of origin. To test, the inventors took a PanCancer approach and k-means clustered RNAseq data from 9,029 solid tumors from all together Gap and silhouette analysis show fifteen PanCancer collagen defined clusters (PanColClusters) was optimal. SevenPanColClusters were homogeneous. The other eight were relatively heterogeneous. The PanColClusters were highly concordant with the twenty-eight iClusters defined by multi-omics by Hoadley et al. (2018). These observations show that collagen expression classified cancer types by their tissues of origin resulting in the same seminal observations as other approaches. Thus, the extracellular matrix characteristics of tumors maintain the features of the tissue of the origin and that the expression of such features including collagens can classify tumors by their tissue of origin.

COL17A1 was highest in the pan-squamous cluster, PanCan-C1, consistent with it being a squamous marker as reported (Jones et al., (2020)). COL20A1 distinguished the brain cancer types (glioblastoma multiforme and brain lower grade glioma) from the other cancer types. COL20A1 is expressed specifically in the brain and testis (F et al., (2014)). The brain cancer types (glioblastoma multiforme and brain lower grade glioma) were grouped into two PanCancer clusters distinguished by IDH1 mutation status, similar to Hoadley et al., (2018)). Even in the relatively low collagen environment of the brain, collagen expression classified tumors into notable and biologically meaningful groups. The gastrointestinal, lung, and breast tumors showed higher expression of fibrillar collagens (PanColClusters C1-C5). PanCanColClusters C6-C15 were defined by combinations of minor collagens. 011, a Pan-Gyn PanCanColCluster, was defined by high expression of COL2A1. PanCanColCluster-C8 was a homogeneous PanCanColCluster for Prostate Adenocarcinoma marked by high expression of both COL2A1 and COL9A2.

Mapping between the PanCanColClusters and ColClusters aids interpretation of the biology of each group across cancer types. the inventors highlight some representative clusters that illustrate how collagen expression distinguishes tissues and histologies across cancer types.

PanCan-C1 is the pan-squamous group and mapped to the three pan-squamous groups identified by Hoadley et al., (2018). They were distinguished by high COL4A5/COL4A6, COL7A1 and COL17A1 expression. Although most lung squamous cell carcinoma tumors were in PanCan-C1:Squamous, LUSC-C4 is a group of lung squamous cell carcinoma tumors that resembles lung adenocarcinoma and mapped to the PanCan-C3:LUAD group. These lung squamous cell carcinoma tumors were characterized by relatively high expression of COL4A3/COL4A4 and relatively lower expression of most other collagens. Bladder urothelial carcinoma was distributed in both the C1:Pan-squamous and the C10:mixed cluster. All tumors in the BLCA-2 ColCluster were in BLCA-C1. C10:mixed mapped to BLCA-C3, BLCA-C4, and BLCA-C5. Collagen expression distinguishes histology features in bladder urothelial carcinoma. The four esophageal carcinoma ColClusters were distributed into the PanCan-C1 squamous group (ESCA-C2 and ESCA-C3) as well as the PanCan-C2:GI and PanCan-C10:mixed group, separating the squamous from the other histologies.

Most kidney tumors were placed into the homogeneous PanCan-C14:KIRP group and PanCan-C15:KIRC group. A few kidney tumors from the KIRC-C3 were distributed among other PanCanColClusters, showing that these tumors differed significantly from the rest of the kidney renal clear cell carcinoma tumors. These same tumors mapped to different iClusters in Hoadley et al., (2018).

Groupings of gynecological cancers reveals similarities in their extracellular matrices. Ovarian serous Cystadenocarcinoma was split into two groups, PanCan-C4 and PanCan-C11. Although Ovarian serous cystadenocarcinoma ColClusters were not associated with overall survival, the high collagen/high stroma OV-C1 group is similar to many sarcoma tumors that have relatively longer overall survival. OV-C2 and OV-C3, clustered with SARC-C4, the SARC group with the shortest overall survival. Thus, the relatively high collagen type I and fibrillar collagens in OV-C1, SARC-C1, and SARC-C2 clustered together with COL2A2, COL4A5/A6 defined OV-C2, OV-C3, and SARC-C4. The sarcomas in are diverse collection of tumors. the inventors further evaluated the tissue of origin and histologies of sarcomas The PanColClusters and ColClusters were strongly enriched for specific sarcoma types and the sarcomas originating from endometrial tissue were grouped with other gynecological cancers.

Collagen expression classified tumors similarly to the whole matrisome gene set. The inventors compared how collagen only clustering corresponded to classifications using hundreds of matrisome genes. These observations show that collagen expression alone captured the seminal features of the classifying tumors based on extracellular matrix features especially those related to overall survival and enrichment of somatic mutations.

The inventors evaluated the relationship between overall mutation rates and microsatellite instability (MSI) with the ColClusters. MSIH tumors were localized to COAD-C1, STAD-C2, and UCEC-C1. Most MSIH cases in stomach adenocarcinoma and colon adenocarcinoma were clustered together. Stomach adenocarcinoma MSIH clusters were marked by collagen types COL10A1 and COL11A1. Notably, a subset of STAD MSS tumors were placed in STAD-C2, with MSIH tumors, because they had similar collagen composition, despite vastly different mutation signatures, showing convergence on extracellular matrix phenotypes originating from distinct genotypes. This is a recurring theme in these data: common extracellular matrix phenotypes associated with a range of genotypes. A group of colon adenocarcinoma MSS tumors was identified with similar collagen composition to colon adenocarcinoma MSI tumors in COAD-C1 and COADREAD-C1. The MSS tumors and MSIH tumors in COAD-C1 and COADREAD-C1 had similar phenotypic characteristics but very different genotypes. Other MSIH tumors were grouped in other colon adenocarcinoma ColClusters and colorectal carcinoma ColClusters with other MSS tumors based on their collagen composition.

Targeting tumors based on molecular alterations is subject to variable responses with often unclear reasons from patient to patient the inventors hypothesized that collagens and the extracellular matrix could indicate contextual differences of the impact of molecular alterations on the tumor. To test these ideas, the inventors evaluated if ColClusters were enriched for the top 50 most frequently mutated genes, as listed in cBioPortal for the 26 cancer types in this study. the inventors also included variants in ABL1, AKT1, AKT2, ALK1, BRCA1, EGFR, ERBB2, FGFR1, FGFR3, FLT3, HRAS, JAK2, KIT, MET, NRAS, PDGFRA, and RET, known critical drivers in some contexts in our analysis. the inventors focused on the most frequent mutations in order to have sufficient numbers to observe biases across the ColClusters. Significance for biased distribution across the ColClusters was determined by a Chi-squared test. Thus, the inventors describe enrichment in specific ColClusters for a few representative examples.

TP53 showed distinct and significantly biased patterns across the ColClusters in bladder urothelial carcinoma (BLCA), breast invasive carcinoma (BRCA), glioblastoma multiforme (GBM), head and neck squamous cell carcinoma (HNSC), brain lower grade glioma (LGG), lung adenocarcinoma (LUAD), sarcoma (SARC), and uterine corpus endometrial carcinoma (UCEC). In each of these cancer types, distinct collagen expression patterns mark the ColClusters, highlighting how the extracellular matrix composition varies for tumors with similar molecular alterations.

There are two general types of patterns observed with gene variants: (1) One ColCluster with strong positive or negative enrichment for a specific molecular alteration relative to the other ColClusters, showing a link between a specific extracellular matrix and a specific molecular alteration. (2) Several ColClusters had similar genetic profiles of candidate drivers or suppressors showing that the genotypes were associated with diverse collagen composition in these settings.

The inventors highlight examples of pattern 1, where specific molecular alterations were localized to one or two ColClusters. PTEN truncations were enriched in all of the uterine corpus endometrial carcinoma ColClusters, except UCEC-C4, which was enriched for P53 missense variants. These patterns highlight the connections between specific genetic features with specific collagen compositions. Wnt signaling in liver tumors is often activated by CNNTB1 mutations. See Perugorria et al. (2019)). Tumors with CNNTB1 mutations were significantly less frequent in LIHC-C1 compared to LIHC-C2 and LIHC-C3, even though the overall mutation rate was not significantly different across these ColClusters. LIHC-C1 is marked by higher fibrillar collagen expression compared to LIHC-C2 and LIHC-C3.

Tumors with IDH1 mutations were enriched in GBM-C3 with all seven tumors with IDH1 variants in GBM-C3. LGG-C1 and LGG-C2 were enriched for IDH1 wild-type tumors and associated with shorter overall survival. These findings show connections between the collagen environment and IDH1/2 mutation status in brain tumors. One of the striking differences between brain lower grade glioma and glioblastoma multiforme is the variation in collagen type IV composition, which is associated with vessel formation in the brain environment. See Lanfranconi & Markus (2010). Brain lower grade glioma tumors had lower COL4A1/2 expression compared to glioblastoma multiforme (GBM). Brain lower grade glioma tumors with relatively higher COL4A1/2 expression compared to other brain lower grade glioma tumors, and also enriched for mutant IDH1/2, may have a distinct vasculature compared to wild-type IDH tumors with lower levels of COL4A1/2 expression. See Huang, Carcinogenesis (2019); Zhang et al., Neuro-Oncology (2018). These findings link vasculature diversity with collagen composition diversity.

Another example of pattern 1 is the distribution of BRAF variants in thyroid carcinoma. Collagen clustering placed BRAF wild-type tumors into THCA-C3, defined by higher COL4A1/COL4A2 expression, along with lower overall survival, and includes only one tumor with mutant BRAF. BRAF mutations in thyroid carcinoma are associated with worse overall survival. See Xing et al. (2014).

Collagen clustering in bladder urothelial carcinoma tumors exemplify pattern 1 for FGFR3 mutations. Mutations in FGFR3 have been associated with less aggressive bladder tumors. Copy number alterations were localized to BLCA-C5, marked by high expression of COL4A5/COL4A6, high expression of COL10A1, with relatively low expression of fibrillar collagens, and the lowest hazard ratios among the five bladder urothelial carcinoma ColClusters. Thus, collagen clustering identified a set of tumors with FGFR3 mutations, with similar overall survival and collagen environments.

Distribution of variants in the breast invasive carcinoma ColClusters exemplifies both patterns. Collagen clustering separated tumors into PIK3CA (BRCA-C1 and BRCA-C3), and TP53 mutation groups (BRCA-C2 and BRCA-C4). BRCA-C1, C3 and C5 were enriched for hormone positive tumors. BRCA-C2 and BRCA-C4 were enriched for Triple Negative Breast Cancers (TNBC). BRCA-C2 and BRCA-C4 have similar collagen type IV levels, but differential expression of collagen type IX and COL2A1. This is an example of Pattern 2, where similar molecular alterations have distinct tumor extracellular matrix composition. Also noteworthy is that many TNBC tumors were classified with hormone positive breast invasive carcinoma tumors because of their common collagen environments.

Genes mutated at a high rate in specific cancer types were distributed in distinct patterns across ColClusters exemplifying pattern 2. ARID1A in uterine corpus endometrial carcinoma, KRAS in colon adenocarcinoma, and TP53 were localized to multiple ColClusters. These ColClusters with similar putative drivers have distinct collagen environments, and different relationships with long and short overall survival.

Variants in tumor suppressors also showed significant bias. Tumors with RB1 truncations were localized to BLCA-4, LUSC-C2/C3, and SARC-C3. RB1 truncations in these tumors were linked to specific collagen environments.

PAAD-C1 had a lower mutation rate, including lower fraction of tumors with mutated KRAS, but this is likely because of the high stroma fraction and lower overall tumor cell percentage in these cases. Re-evaluation of the rate of KRAS mutation in showed the expected high rate of KRAS mutations that were missed in. Raphael et al., (2017). It is of note that PAAD-C1, defined by high fibrillar collagen expression, was associated with a lower mutation rate, and had only a modest difference in stroma fraction compared to the other ColClusters.

The inventors evaluated if the top fifty most common gene copy number alterations (CNAs) observed in the twenty-six cancer types were biased across the ColClusters using the copy number calls provided. Gene level copy number aberrations showed distinct distributions among the ColClusters in all cancer types except colon adenocarcinoma. Amplifications of Myc showed a biased distribution in ten cancer types. Notably, Myc amplifications were not enriched in most ColCluster-1's, except for liver hepatocellular carcinoma and ovarian serous cystadenocarcinoma. 86% of Testicular Germ Cell Tumors showed copy gains for KRAS. KRAS copy gain was negatively enriched in TGCT-C1.

Notably, even though the three ovarian serous cystadenocarcinoma ColClusters have similar overall aneuploidy, specific copy number alterations were distinct in OV-C1 and OV-C2 compared to OV-C3. OV-C3 was enriched for SOX2 copy gains. OV-C1 was enriched for AGO2, MYC and RAD21 copy gains. Collagen classification of ovarian serous cystadenocarcinoma tumors identified specific tumor groups linking copy number alterations with extracellular matrix context. OV-C1 and OV-C2 were significantly enriched for gains in MYC. OV-C3 was enriched for CDK4 and KRAS. EGFR copy gains were significantly biased in nine cancer types including in glioblastoma multiforme.

Tumor suppressors such as the cell cycle regulators, CDNK2A and MTAP, showed copy number losses in specific ColClusters including GBM-C1 and C4, ESCA-C2 and C4, and BLCA-C5. SARC-C1 was enriched for MDM2, CCNE1, and CDK4 gains. These findings reveal connections between molecular alterations controlling the cell cycle and the collagen environment.

Chromosome level copy number alterations are strong markers for both diagnosis and prognosis in many cancer types. the inventors investigated the relationships between specific chromosome arm copy number alterations and collagen expression. the inventors evaluated chromosome arm copy number alterations with at least ten copy number alterations in the cancer type. The distribution of many chromosome arm copy number alterations was significantly biased across ColClusters in many tumor settings as assessed by a Chi-squared test. ColClusters enriched for three copy number alterations across multiple chromosomes were observed in breast invasive carcinoma (BRCA), esophageal carcinoma (ESCA), head and neck squamous cell carcinoma (HNSC), kidney renal clear cell carcinoma (KIRC), kidney renal papillary cell carcinoma (KIRP), stomach adenocarcinoma (STAD), thyoma (THYM), and uterine corpus endometrial carcinoma (UCEC). Some ColClusters revealed high levels of both gains and losses including: STAD-C3, STAD-C5, THYM-C3, UCEC-C4, LUAD-C3, LIHC-C2, and COAD-C3. Others were biased towards gains or losses including PAAD-C4, BRCA-C2 and BRCA-C4, and KIRP-C3.

Chromosome arm level copy number alterations were localized to a specific ColCluster in many cancer types including endocervical adenocarcinoma (1q gain), colon adenocarcinoma (1p loss), glioblastoma multiforme (9p loss), head and neck squamous cell carcinoma (11q loss), brain lower grade glioma (1q gain, 19q loss), pancreatic adenocarcinoma (17p, 18q gains), pancreatic adenocarcinoma (3p loss) and sarcoma (10q loss). Some chromosome arm-level copy number alterations were strongly biased across the ColClusters. The distribution of 3p loss was significantly biased in several cancer types including breast invasive carcinoma, bladder urothelial carcinoma, esophageal carcinoma, head and neck squamous cell carcinoma, lung squamous cell carcinoma, and stomach adenocarcinoma. 90% of kidney renal clear cell carcinoma tumors have human chromosome 3p loss, but those that do not are almost all in KIRC-C3. These connections between specific chromosome arm level copy number alterations and the extracellular matrix may provide clues to the genetic adaptations required to remodel and to create specific extracellular matrix environments for tumor cells to succeed compared to other cells.

ColClusters in cancer types such as esophageal carcinoma showed specific enrichment patterns. ESCA-C2 was enriched for human chromosome 8p gains. ESCA-C1 and ESCA-C3 were enriched for human chromosome 18q losses. ESCA-C2 and ESCA-C4 had many copy number gains but no aneuploidy/ploidy distribution differences. Human chromosome 10p loss was enriched in LGG-C1 and LGG-C2 while human chromosome 19q loss was enriched only in LGG-C5. Thus, the existence of specific relationships between collagen expression and chromosome copy number aberrations linking the cancer genome with the tumor extracellular matrix. Ovarian serous cystadenocarcinoma ColClusters and lung squamous cell carcinoma ColClusters have similar distribution of copy number alterations, likely because most of these tumors harbor the same copy number alterations.

To test for specific relationships between chromosome arm copy number aberrations and collagen expression, the inventors implemented a Support Vector Machine model to predict chromosome arm copy number aberration status based solely on collagen mRNA expression. the inventors tested the quality of the model by five-fold cross validation in each cancer type with ten cases. the inventors used the area under the curve (AUC) of the receiver operating characteristic (ROC) to evaluate the model performance in each tumor setting. The Support Vector Machine model predicted human chromosome 3p loss in 59% of the cancer types with at least ten cases with human chromosome 3p loss. This shows that collagen composition is strongly linked to human chromosome 3p loss in multiple cancer settings. the inventors extended this analysis to all the chromosome arms as summarized. Human chromosome 5q and 9q losses were predicted very well in multiple cancer types. These connections show potential genetic adaptations required to thrive in specific extracellular matrix environments.

These observations of the copy number alterations and structural variations show associations between ploidy, genome doublings, and aneuploidy in the ColClusters. Aneuploidy has been associated with a range of treatment responses and patient survival risk depending on contexts. See Vasudevan et al., (2021); Ben-David & Amon (2020)). The inventors evaluated the relationship between aneuploidy and the collagen defined clusters. 12 cancer types showed significantly altered distribution across the ColClusters as assessed by a Kolmogorov-Smirnov (KS) test. Bladder urothelial carcinoma, colon adenocarcinoma, lung adenocarcinoma, stomach adenocarcinoma, and uterine corpus endometrial carcinoma cancer types showed very strong biases with the majority of high or low aneuploid tumors grouped into one or two ColClusters.

In stomach adenocarcinoma, two ColClusters, STAD-C3 and STAD-C5, with relatively high aneuploidy were identified, but with strikingly different overall survival and collagen expression patterns. The median overall survival for the high aneuploidy tumors in STAD-C3 is 14.4 months compared to 37.5 months for the high aneuploidy tumors in STAD-C5. UCEC-C4 is enriched for high aneuploidy tumors, but many high aneuploidy tumors were distributed across the other three uterine corpus endometrial carcinoma ColClusters. These observations show that the high aneuploidy tumors in UCEC-C4 are a distinct set of aggressive high aneuploidy tumors with different collagen composition, where patients have particularly short overall survival, compared to the other high uterine corpus endometrial carcinoma aneuploid tumors. These observations show that the combination of aneuploid and collagen composition may explain some of the confounding observations where aneuploidy is not always associated with worse outcomes. See Taylor et al. (2018).

To explore the relationship between collagen expression and aneuploidy further, we used a Support Vector Machine model to test if collagen expression can predict aneuploidy levels in tumors. The inventors modeled the aneuploidy scores with Gaussians to partition the scores into high and low categories. The Support Vector Machine predicted the aneuploidy status of 9 of the cancer types with area under the curves (AUC) 0.8 by Receive Operator Characteristic (ROC) analysis. Evaluation of the weights for each collagen reveal that each cancer type has specific collagen expression patterns. Similar performances of Support Vector Machine models were observed for the related metrics, i.e., genome doublings and ploidy.

The inventors compared the Support Vector Machine predictions of aneuploid levels from collagen expression to the ColCluster-aneuploidy enrichments. Some cancer types, including liver hepatocellular carcinoma, ovarian serous cystadenocarcinoma and esophageal carcinoma, did not show biased distribution of aneuploidy scores in the ColClusters. But the Support Vector Machine accurately predicted aneuploidy levels in ovarian serous cystadenocarcinoma showing a relationship between collagen expression and aneuploidy. Other cancer types such as sarcoma and uterine corpus endometrial carcinoma showed ColCluster enrichments with reasonable Support Vector Machine predictions with AUCs of 0.73 and 0.74, respectively, just below the 0.75 threshold.

These observations show a relationship between the cancer genome and collagen expression. They further imply that not all aneuploid tumors have similar features and that the combination of aneuploidy and the extracellular matrix should be considered to understand tumor progression and therapeutic options.

The tumor extracellular matrix is a critical regulator of immune cell infiltration through myriad mechanisms including mechanical blockage (Leight et al., (2016)), angiogenesis by basement membrane collagens (Sekiguchi & Yamada (2018)), or stimulation of specific signaling pathways (Leight et al. (2016)). Enrichment of immune cell expression signatures derived from Tamborero et al., (2018)) were determined by QuSAGE to identify the ColClusters enriched for each cell type compared to the other ColClusters. See Meng et al., (2019). Regulatory T-cells and macrophages were enriched in many of the high stroma ColCluster 1's. 9/26 ColCluster 1's were highest for T-regs compared to the other ColClusters, showing connections between these immunosuppressive cells and tumors with high expression of fibrillar collagen. These observations are relative observations and identify classes of tumors to consider for more traditional therapy and immunotherapy responses.

STAD-C1 and C2 have similar stroma fractions, but significantly different immunoenvironments. STAD-C1 may be more immunosuppressive with higher Treg infiltration, while STAD-C2 may be more immune activated with enrichment for activated dendritic cells (aDCs) and higher expression of inflammatory gene signatures, consistent with STAD-C2 associated with longer overall survival.

BLCA-C1 and BLCA-C2 have similar levels of stroma fraction, as well as expression of many of the fibrillar collagens, but showed distinct immune cell infiltration patterns. BLCA-C1 was enriched for activated CD8 T cells, B cells and regulatory T cells while BLCA-C2 was enriched for aDC cells. These observations connect specific collagen defined tumor classes with immune cell infiltration patterns.

To assess the global immunoenvironment in each ColCluster, we identified significant biased distributions for the six immunotypes defined by Thorsson et al., Immunity (2018) in all but two cancer settings. BRCA-C2 and BRCA-C4 were enriched for the “IGFN-γ” immune group, similar to all three ovarian serous cystadenocarcinoma ColClusters, and UCEC-C4. These groups have high levels of structural variations with high aneuploidy levels. LGG-C2 had a more GBM-like immunoenvironment as it is enriched for “C4-lymphocyte depleted” compared to the large majority of tumors placed in immunotype-C5, “immuno-logically quiet” in the other four brain lower grade glioma ColClusters. LUAD-C3 and LUAD-C4 were enriched for immunotype-C3, “Inflammatory”, while the other LUAD ColClusters were enriched for immunotypes LUAD-C1 and LUAD-C2. LUSC-C4 was biased to immunotype LUAD-C2, while the others were divided between immunotypes C1 and C2. Uterine corpus endometrial carcinoma showed a distinct pattern with immunotype C2, “IFNγ dominant”, strongly enriched in the high aneuploidy UCEC ColCluster-4, while the other three uterine corpus endometrial carcinoma ColClusters were biased towards immunotype LUAD-C1, “Wound Healing”. ColClusters for liver hepatocellular carcinoma and skin cutaneous melanoma had a distinct difference in immunotypes. In some cancer types, the same immunotype was observed in multiple ColClusters including colon adenocarcinoma (COAD), colorectal carcinoma (COADREAD), glioblastoma multiforme (GBM), brain lower grade glioma (LGG), prostate adenocarcinoma, STAD-C4, STAD-C5, and thyroid carcinoma (THCA). In other cancer types, including bladder urothelial carcinoma and breast invasive carcinoma, the distribution of immunotypes was similar across all the ColClusters with only subtle biases observed. The high aneuploidy ColClusters, including STAD-C3 and UCEC-C4 were enriched for distinct immunotypes relative to the other stomach adenocarcinoma ColClusters and uterine corpus endometrial carcinoma ColClusters. These observations show that collagen composition was associated with specific immunoenvironment.

To assess the biological features enriched in each ColCluster, the fifty Molecular Signature Database (MSigDB) cancer hallmark gene sets were evaluated using QuSAGE, which identified the ColClusters where each gene set is most enriched relative to the other ColClusters. We examined patterns to determine which ColClusters were most enriched for hallmarks. Thirteen cancer types had at least one ColCluster enriched for ten hallmarks.

Increased collagen type I secretion has been associated with TGFβ signaling and epithelial mesenchymal transition in several models. Xu (2009). The inventors examined the relationship between the high stroma fraction ColCluster-1's and these hallmarks. 13/26 ColCluster-1's were highest in TGFβ signaling. TGFβ and epithelial mesenchymal transition (EMT), in particular, were associated with expression of fibrillar collagens and high stroma ColClusters. Epithelial mesenchymal transition was highest in ColCluster-1's including bladder urothelial carcinoma (BLCA), endocervical adenocarcinoma, colon adenocarcinoma (COAD), colorectal carcinoma (COADREAD), glioblastoma multiforme (GBM), head and neck squamous cell carcinoma (HNSC), kidney renal clear cell carcinoma (KIRC), kidney renal papillary cell carcinoma (KIRP), liver hepatocellular carcinoma (LIHC), lung adenocarcinoma (LUAD), lung squamous cell carcinoma (LUSC), ovarian serous cystadenocarcinoma (OV), rectal adenocarcinomas (READ), stomach adenocarcinoma (STAD), thyroid carcinoma (THCA), and uterine corpus endometrial carcinoma (UCEC). ESCA-C4, LGG-C2, PAAD-C2, PCPG-C2, SARC-C2, TCGT-C4, THYM-C3, were relatively high in epithelial mesenchymal transition and fibrillar collagen gene expression in these cancer types. Several collagens promote angiogenesis including collagen types I and IV. See Leight et al. (2016). The angiogenesis hallmark gene set was associated with the ColCluster with high collagen type I and fibrillar collagen expression in nineteen cancer types.

Not all hallmark gene sets were associated with high fibrillar collagen expression. Many hallmarks showed specific patterns across the ColClusters and were enriched in other ColClusters. Bile acids may decrease adhesion to collagens. Bile acid metabolism with the highest QuSAGE values in each cancer type is enriched in ColClusters other than the high fibrillar collagen ColClusters, except for BRCA-C3, KIRP-C1, and TGCT-C4. For many cancer types, high levels of bile acid metabolism were associated with relatively high fibrillar collagen environments. ColClusters including BRCA-C2, BRCA-C4, STAD-C3 had relatively high expression of the Myc target gene set, consistent with Myc amplification in these clusters. These observations show distinct pathways are active in each ColCluster connecting the collagen environment to targetable processes.

Collagen clustering and the Support Vector Machine model show relationships between aneuploidy and the extracellular matrix. To test the impact of high and low aneuploid tumors in different extracellular matrix contexts, we stratified tumors by high and low aneuploidy to examine the relationship with overall survival. In multiple cancer types, aneuploidy was associated with overall survival in specific ColClusters, but not others, even when the number of cases was significant. Two types of patterns are observed: high aneuploid tumors were grouped separately from low aneuploidy tumors. In other examples, high and low aneuploidy tumors have similar collagen environments and were grouped in the same ColCluster. They have very different associations with overall survival relative to other high/low aneuploidy tumors in the other ColClusters.

Bladder urothelial carcinoma tumors with high aneuploidy were separated by overall survival by collagen composition, while the low aneuploid BLCA tumors were not separated by overall survival. A large difference in overall survival between high and low aneuploid tumors was observed for BLCA-C4, the lowest overall survival bladder urothelial carcinoma ColCluster. BLCA-C4 is marked by a combination of COL2A1, COL4A3, and COL11A2 among others. Similar observations were made for liver hepatocellular carcinoma, driven by the large difference in overall survival in LIHC-C3 between high and low aneuploid tumors.

Lung squamous cell carcinoma exemplifies context dependent aneuploidy as patients with high aneuploid tumors in LUSC-C4 have relatively lower risk while patients with high aneuploid tumors in LUSC-C5 have higher risk. High aneuploid uterine corpus endometrial carcinoma tumors have lower overall survival, which trends in all the ColClusters. The most extreme example is UCEC-C4, which has the overall shortest survival, which is driven by the high aneuploid tumors. Within the same collagen extracellular matrix environment, low aneuploid tumors have very low risk in UCEC-C4, highlighting the extreme differences in adaptation within the same extracellular matrix environment.

ColClusters mapped to specific PanClusters showing that the tumors in these ColClusters differ in their tissue and histology characteristics and were grouped to different cancer types. Integrating these observations with the other analyses presented in this study reveals new insights into the tumors. For example, the high aneuploidy ColClusters, with relatively short overall survival, STAD-C3, UCEC-C4, SARC-C4, were grouped together in the Pan-Gyn, PanCan-C11 group, along with BRCA-C2 and BRCA-C4, characterized by many copy number gains. Conversely, the longer overall survival STAD-C4 group mapped to the heterogeneous PanCan-C10 group with BLCA-C3, BLCA-C5, ESCA-C3, OV-C2, OV-C3, and KIRP-C3; all with relatively lower levels of aneuploidy, marked by collagen type IX expression with lower fibrillar collagen expression. Thus, classes of tumors originating from a range of tissues had high aneuploidy and similar collagen composition. Conversely, a group of ColClusters in gastrointestinal (GI) tumors were enriched for tumors with lower levels aneuploidy, but also had relatively short overall survival, including STAD-C1, COAD-C1, and PAAD-C1. These ColClusters have relatively high expression of fibrillar collagens. Here, we highlight a few ColClusters where combining the genetics, environment, and collagen composition clustering reveals new opportunities for therapeutic and biomarker development.

STAD-C5 included a mixture of tumors with high and low aneuploidy classified together with similar collagen expression profiles. These tumors were enriched for Wnt Beta Catenin signaling hallmarks. STAD-C5 had longer overall survival compared to the other ColClusters. GBM-C3 is enriched for proliferation gene sets including E2F targets and G2M cell cycle as well as the Wnt Beta Catenin hallmark gene set.

BLCA-C1 and BLCA-C2 have similar expression of the fibrillar collagens and stroma fraction. BLCA-C2 is marked by COL17A1 expression and includes many squamous tumors. BLCA-C1 is enriched for EMT and angiogenesis hallmark gene sets while BLCA-C2 was enriched for twenty-seven hallmark gene sets compared to four gene sets in BLCA-C1 with five gene sets with similar QuSAGE scores. BLCA-C5 is enriched for FGFR3 mutations and is highest for Notch hallmark gene sets. Notch may be a tumor suppressor pathway and is consistent with patients in BLCA-C5 having the longest overall survival BLCA-C3 and BLCA-C4, distinguished by several minor collagens and relatively lower levels of fibrillar collagen expression. BLCA-C3 was enriched for bile acid metabolism. BLCA-C4 was enriched for cell cycle regulation and had the shortest overall survival among the bladder urothelial carcinoma ColClusters.

The high aneuploidy UCEC-C4 cluster is enriched for Notch signaling along with DNA repair and proliferation gene sets showing possibilities for therapeutic development in this class of tumor defined by combinations of genetics and collagen composition.

### FURTHER EMBODIMENTS

Specific compositions and methods for classifying tumors by their collagen expression patterns into groups associated with high and low overall survival. The scope of the invention should be defined solely by the claims. A person having ordinary skill in the biomedical art will interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

Further embodiments of the invention include the following:

A method for treating cancer in a subject, comprising the steps of (a) selecting a tumor classification associated with high and low overall survival for a tumor by its collagen expression patterns into groups; and (b) treating the subject with a cancer treatment specific for the tumor classification associated with high and low overall survival:

wherein the specific cancer genomes are noted by features such as somatic mutations, ploidy, and aneuploidy; or

wherein connections with hallmarks indicate links between therapy responses and options based on collagen composition; or

wherein the collagen expression patterns identify tumors that differ from normal tissue through dsyregulation of specific collagens and high expression of COL1A1 and fibrillar collagens (COL5, COL11, COL14); or

wherein the selecting considers the extracellular matrix and the major component of the ECM, collagens, helps predict patient outcomes; or

wherein collagen mRNA expression robustly classifies tumors and identifies tissues of origin; or

wherein collagen based clusters associate with overall survival; or

wherein tumors with collagen type I and fibrillar collagen expression have relatively lower aneuploidy levels for example compared to collagen defined groups with other collagens; or

wherein the collagens define the squamous histologies in bladder and esophageal tumors, demonstrating the power of collagen lineage and histology connections to classify tumors; or

wherein collagen-defined clusters are enriched with cancer hallmarks; or

wherein stratifying patients by combinations of collagen composition (ColClusters) and molecular alterations such as aneuploidy identifies connections with longer or shorter overall survival; or

wherein collagen defined clusters are associated with overall survival.

A machine learning that demonstrates the connections between collagen expression and molecular alterations. Collagen expression predicts molecular alterations. This highlights the phenotypic environment and the genomic features being selected. Integrating collagen classification with molecular alterations, immunotypes and cancer hallmarks identifies tumor classes to target.

Machine learning predicts genomic features. This is an important finding linking the collagen composition with the presence of specific molecular alterations in the tumor genomes.

Collagen tumor patterns are distinct from normal tissue ECM and collagen expression patterns. Collagens are dysregulated in tumors. Many collagens including COL10A1 and COL7A1 have very specific expression in normal healthy tissue. But these two collagens are then dysregulated and expressed in both stroma, fibroblast cells and/or cancer cells in tumors.

Collagen clusters are associated with specific cancer genomes. The specification shows that collagen clusters are enriched for specific molecular alterations including point mutations of many cancer drivers and suppressors.

Clusters are enriched for specific mutation patterns. This clustering is the primary enrichment. The subsequent association with overall survival indicates the treatments in the TCGA patients.

Clusters are enriched for specific copy number alterations. Copy number alterations can be targeted specifically with certain drugs. Genes with high or low copy number indicate therapy options. Putting together with collagen and the ECM composition refines the potential drug responses is the idea here. It is the combination of considering the local environment i.e., the ECM and collagen composition, with the molecular features such as gene copy number.

Enrichment and the machine learning demonstrate relationship between collagen composition and aneuploidy, ploidy and genome doubling. As shown in FIG. 7 and in some references, aneuploidy in primary tumors has unclear relationship with drugs responses and overall survival. When combined with specific collagen composition tumors, some collagen defined tumor groups combined with aneuploidy are now associated very strongly with overall survival. This shows how considering the tumor collagen ecosystem and the extracellular matrix together with aneuploidy identifies patients/tumors that do poorly or better.

Each of the clusters has distinctive associations with overall survival and help link the molecular alterations with outcomes, which is better than just considering the molecular alterations by themselves. Many of these molecular alterations are not cleanly associated with outcomes in many cancer types. Considering the collagen composition and combining with the molecular alteration makes a big difference and improves the prediction of the outcomes and association with overall survival.

### CITATION LIST

A person having ordinary skill in the biomedical art can use these patents, patent applications, and scientific references as guidance to predictable results when making and using the invention.

### NON-PATENT LITERATURE

Brodsky et al., Classification of tumors by collagen expression reveals genotype-tumor ECM interactions [abstract]. In: Proceedings of the AACR virtual special conference on the evolving tumor microenvironment in cancer progression: Mechanisms and emerging therapeutic opportunities; in association with the tumor microenvironment (TME) Working Group; 2021 Jan. 11-12. Philadelphia (Pa.): AACR; Cancer Res 81(5 Suppl), Abstract nr P0019 (2021).
Hoadley et al., Cell-of-origin patterns dominate the molecular classification of 10,000 tumors from 33 types of cancer. Cell, 173(2) (Apr. 5, 2018).
Ben-David & Amon, Context is everything: Aneuploidy in cancer. Nature Reviews Genetics, 21(1), 44-62 (2020).
Brodsky et al., Expression profiling of primary and metastatic ovarian tumors reveals differences indicative of aggressive disease. PLoS ONE, 9(4), e94476 (2014).
Brodsky et al., Identification of stromal ColX1 and tumor-infiltrating lymphocytes as putative predictive markers of neoadjuvant therapy in estrogen receptor-positive/HER2-positive breast cancer. BMC Cancer, 16(1), 274 (2016).
Brodsky et al., Classification of tumors by collagen expression reveals genotype-tumor ECM interactions [abstract]. In: Proceedings of the AACR Virtual Special Conference on the Evolving Tumor Microenvironment in Cancer Progression: Mechanisms and Emerging Therapeutic Opportunities; in association with the Tumor Microenvironment (TME) Working Group; 2021 Jan. 11-12. Philadelphia (Pa.): AACR; Cancer Res., 81(5 Suppl), Abstract nr P0019 (2021).
Busslinger et al., Human gastrointestinal epithelia of the esophagus, stomach, and duodenum resolved at single-cell resolution. Cell Reports, 34(10), 108819 (2021).
Cosgrove et al., Collagen COL4A3 knockout: A mouse model for autosomal Alport syndrome. Genes & Development, 10(23), 2981-2992 (1996).
Engel, Cress & Santiago-Cardona, The retinoblastoma protein: A master tumor suppressor acts as a link between cell cycle and cell adhesion. Cell Health and Cytoskeleton, 7, 1-10 (2014).
Fagerberg et al., Analysis of the human tissue-specific expression by genome-wide integration of transcriptomics and antibody-based proteomics*. Molecular & Cellular Proteomics, 13(2), 397-406 (2014).
Farmer et al., A stroma-related gene signature predicts resistance to neoadjuvant chemotherapy in breast cancer. Nature Medicine, 15(1), 68-74 (2009).
Feng et al., Lgr5 and Col22a1 mark progenitor cells in the lineage toward juvenile articular chondrocytes. Stem Cell Reports, 13(4), 713-729 (2019).
Hoadley et al., Isocitrate dehydrogenase mutations in glioma: From basic discovery to therapeutics development. Frontiers in Oncology, 9,506 (2019).
Huang, Friend or foe-IDH1 mutations in glioma 10 years on. Carcinogenesis, 40(11), 1299-1307 (November 2019).
Izzi et al., Pan-cancer analysis of the expression and regulation of matrisome genes across 32 tumor types. Matrix Biology Plus, 1,100004 (2019).
Jones et al., The role of collagen XVII in cancer: Squamous cell carcinoma and beyond. Frontiers in Oncology, 10,352 (2020).
Junker et al., Fibroblast growth factor receptor 3 mutations in bladder tumors correlate with low frequency of chromosome alterations. Neoplasia, 10(1), 1-7 (2008).
Kastenhuber & Lowe, Putting p53 in context. Cell, 170(6), 1062-1078 (2017).
Lanfranconi & Markus, COL4A1 Mutations as a monogenic cause of cerebral small vessel disease. Stroke, 41(8), e513-e518 (2010).
Leight, Drain, & Weaver, Extracellular Matrix Remodeling and Stiffening Modulate Tumor Phenotype and Treatment Response. Annual Review of Cancer Biology, 1(1), 313-334 (2016).
Letai, Bhola, & Welm, Functional precision oncology: Testing tumors with drugs to identify vulnerabilities and novel combinations. Cancer Cell, 40(1), 26-35 (2021).
Lindgren et al., (2021). Type IV collagen as a potential biomarker of metastatic breast cancer. Clinical & Experimental Metastasis, 38(2), 175-185.
Liu et al., Stem cell competition orchestrates skin homeostasis and ageing. Nature, 568(7752), 344-350 (2019).
Meng et al., Gene set meta-analysis with Quantitative Set Analysis for Gene Expression (QuSAGE). PLOS Computational Biology, 15(4), e1006899 (2019).
Naba et al., Characterization of the extracellular matrix of normal and diseased tissues using proteomics. Journal of Proteome Research, 16(8), 3083-3091 (2017).
Nallanthighal, Heiserman, & Cheon, Collagen type XI alpha 1 (COL11A1): A Novel Biomarker and a Key Player in Cancer. Cancers, 13(5), 935 (2021).
Perugorria et al., Wnt-catenin signalling in liver development, health and disease. Nature Reviews Gastroenterology & Hepatology, 16(2), 121-136 (2019).
Phelan et al., Bile acids destabilise HIF-1 and promote anti-tumour phenotypes in cancer cell models. BMC Cancer, 16(1), 476 (2016).
Pickup, Mouw, & Weaver, The extracellular matrix modulates the hallmarks of cancer. EMBO reports, 15(12), 1243-1253 (2014).
Raphael et al., (2017). Integrated Genomic Characterization of Pancreatic Ductal Adenocarcinoma. Cancer cell, 32(2), 185-203.e13.
Ricard-Blum, The collagen family. Cold Spring Harbor Perspectives in Biology, 3(1), a004978 (2011).
Sekiguchi & Yamada, Basement membranes in development and disease. Current Topics in Developmental Biology, 130, 143-191 (2018).
Shen, The role of type X collagen in facilitating and regulating endochondral ossification of articular cartilage. Orthodontics & Craniofacial Research, 8(1), 11-17 (2005).
Tamborero et al., A pan-cancer landscape of interactions between solid tumors and infiltrating immune cell populations. Clinical Cancer Research, 24(15), 3509 (2018).
Taylor et al., Genomic and functional approaches to understanding cancer aneuploidy. Cancer Cell, 33(4), 676-689.e3 (2018).
Thorsson et al., The immune landscape of cancer. Immunity, 48(4), 812-830.e14 (Apr. 17, 2018) is a review article that provides aneuploidy score, stromal fraction, and mutation rate data.
Tian et al., Association Between BRAF V600E Mutation and Recurrence of Papillary Thyroid Cancer. Journal of Clinical Oncology, 33(1), 42-50 (2014).
Zhang et al., IDH mutation status is associated with distinct vascular gene expression signatures in lower-grade gliomas. Neuro-Oncology, 20(11), 1505-1516 (2018).

### TEXTBOOKS AND TECHNICAL REFERENCES

Current Protocols in Immunology (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).
Current Protocols in Molecular Biology (CPMB), (2014). Frederick M. Ausubel (ed.), John Wiley and Sons (ISBN 047150338X, 9780471503385).
Current Protocols in Protein Science (CPPS), (2005). John E. Coligan (ed.), John Wiley and Sons, Inc.
Immunology (2006). Werner Luttmann, published by Elsevier.
Janeway's Immunobiology, (2014). Kenneth Murphy, Allan Mowat, Casey Weaver (eds.), Taylor & Francis Limited, (ISBN 0815345305, 9780815345305).
Laboratory Methods in Enzymology: DNA, (2013). Jon Lorsch (ed.) Elsevier (ISBN 0124199542).
Lewin's Genes XI, (2014). published by Jones & Bartlett Publishers (ISBN-1449659055).
Molecular Biology and Biotechnology: a Comprehensive Desk Reference, (1995). Robert A. Meyers (ed.), published by VCH Publishers, Inc. (ISBN 1-56081-569-8).
Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).
The Encyclopedia of Molecular Cell Biology and Molecular Medicine, Robert S. Porter et al., (eds.), published by Blackwell Science Ltd., 1999-2012 (ISBN 9783527600908).
The Merck Manual of Diagnosis and Therapy, 19^{th} edition (Merck Sharp & Dohme Corp., 2018).
Pharmaceutical Sciences 23^{rd} edition (Elsevier, 2020).

1. A method for treating cancer in a subject, comprising the steps of
(a) selecting a tumor classification associated with high and low overall survival for a tumor by its collagen expression patterns into groups; and
(b) treating the subject with a cancer treatment specific for the tumor classification associated with high and low overall survival.

2. The method of claim 1, wherein the tumor is selected from the group consisting of bladder urothelial carcinoma (BLAC); breast invasive carcinoma (BRAC); endocervical adenocarcinoma (CESC); colon adenocarcinoma (COAD); colorectal carcinoma (COADREAD); esophageal carcinoma (ESCA); glioblastoma multiforme (GBM); head and neck squamous cell carcinoma (HNSC); kidney renal clear cell carcinoma (KIRC); kidney renal papillary cell carcinoma (KIRP); brain lower grade glioma (LGG); liver hepatocellular carcinoma (LIHC); lung adenocarcinoma (LUAD); lung squamous cell carcinoma (LUSC); ovarian serous cystadenocarcinoma (OV); pancreatic adenocarcinoma (PAAD); pheochromocytoma and paraganglioma (PCPG); prostate adenocarcinoma (PRAD); rectal adenocarcinomas (READ); sarcoma (SARC); skin cutaneous melanoma (SKCM); stomach adenocarcinoma (STAD); testicular germ cell tumors (TGCT); thyroid carcinoma (THCA); thyoma (THYM); and uterine corpus endometrial carcinoma (UCEC).

3. The method of claim 2, wherein the specific cancer genomes are noted by features such as somatic mutations, ploidy, and aneuploidy.

4. The method of claim 2, wherein connections with hallmarks indicate links between therapy responses and options based on collagen composition.

5. The method of claim 1, wherein stratifying patients by combinations of collagen composition (ColClusters) and molecular alterations identifies connections with longer or shorter overall survival.

6. The method of claim 1, wherein the collagen expression patterns identify tumors that differ from normal tissue through dsyregulation of specific collagens and high expression of COL1A1 and fibrillar collagens (COL5, COL11, COL14).

7. The method of claim 1, wherein the collagen expression patterns define the squamous histologies in bladder and esophageal tumors.

8. The method of claim 1, wherein the treatment comprises targeting pathways selected form the group consisting of DNA repair, E2F, and Myc in BRCA-C2 and BRCA-C4.

9. A machine learning classifier that predicted a tumor's aneuploidy, KRAS mutation, Myc amplification or chromosome arm copy number alteration (CNA) status based on only collagen RNA expression with high accuracy in many cancer types.
